(19) United States
(12) Patent Application Publication
(10) Pub. No.: US 2026/0053849 A1
(43) Pub. Date: Feb. 26, 2026

(54) NEW IRON AND ACACIA GUM ORAL COMPOSITIONS, PROCESS FOR THEIR PREPARATION AND THEIR USE IN IRON DEFICIENCY CONDITIONS

(71) Applicant: PHARMANUTRA S.P.A, PISA (IT)

(72) Inventors: Andrea LACORTE, PISA (IT); Germano TARANTINO, PISA (IT); Elisa BRILLI, PISA (IT)

(21) Appl. No.: 19/153,872

(22) PCT Filed: Feb. 7, 2024
(86) PCT No.: PCT/IB2024/051129

§ 371 Date: Aug. 5, 2025

(30) Foreign Application Priority Data
Feb. 7, 2023 (IT) 102023000001983
Feb. 7, 2023 (IT) 102023000001989
Feb. 7, 2023 (IT) 102023000001998

(51) Int. Cl. A61K 33/42; A61K 47/02; A61K 47/24; A61K 47/36; A61P 3/12

(52) U.S. Cl. CPC A61K 33/42; A61K 47/02; A61K 47/24; A61K 47/36; A61P 3/12

(57) ABSTRACT

The present invention relates to novel iron pharmaceutical and/or nutraceutical and/or food compositions and their use in the treatment and/or prevention of conditions of absolute or relative iron deficiency in individuals in need. Specifically, the present invention relates to novel compositions comprising highly assimilable iron and their use in the treatment and/or prevention of disorders and conditions associated with iron deficiency. The invention also relates to processes for the preparation of said compositions.

The present invention relates to novel iron pharmaceutical and/or nutraceutical and/or food compositions and their use in the treatment and/or prevention of conditions of absolute or relative iron deficiency in individuals in need. Specifically, the present invention relates to novel compositions comprising highly assimilable iron and their use in the treatment and/or prevention of disorders and conditions associated with iron deficiency. The invention also relates to processes for the preparation of said compositions.

In conditions of absolute or relative iron deficiency in the body, iron (iron (II) or iron (III)) administration is used orally or, in the most severe cases, parenterally. Such conditions include sideropenic anemias and iron supplementation during pregnancy, when necessary.

Although oral administration is preferred, however, iron supplementation through this route of administration has significant drawbacks and limitations. In fact, administration of the classical oral compositions based on ferrous sulfate or gluconate (iron (II)), leads to poor absorption of the said iron salts due to their poor bioavailability. In addition to this, oral administration of iron can cause constipation and stomach pain, and, in the most severe cases, peptic ulcer, gastritis and ulcerative colitis. For this reason, such compositions are generally taken together with meals, a fact that leads to a consequent and inevitable further high reduction of iron absorption in the gastrointestinal tract.

Because of these drawbacks, it is necessary to prolong iron salts therapy over time, up to even 3-6 months until iron stores in the body are adequately restored.

Considering the limitations and side effects described above, also often associated with an unpleasant odor and taste of the compositions due to the easy degradation of the said ferrous salts, it is understood that classical ferrous sulfate or gluconate therapy is not favorable for patient compliance.

Iron (III) salts are also known and commercially available, which, although less soluble in water and less bioavailable than iron (II) salts, have the advantage of being more stable and consequently do not exhibit organoleptic changes even when said iron (III) salts are mixed with other components or ingredients to form a final composition.

Among the iron (III) salts is iron (III) pyrophosphate, which is commercially available in various compositions and varying degrees of hydration. Some of these compositions may sometimes present a difficulty in formulation and show a tendency to give insoluble precipitates that could make their dosing and use difficult.

Patent application CN101455401 describes a multifunctional composition to strengthen the iron, zinc and calcium content that stabilizes a liquid milk system; said composition (embodiment 3) is 100 kg total weight and comprises, among others, 4% Iron (III) pyrophosphate, 13.5% sucrose ester and 6% sodium pyrophosphate.

Scientific article XP055661417 by Colin I. Cercamondi et al. suggests that sodium pyrophosphate might have an effect on the absorption of iron present in broth cubes fortified with iron pyrophosphate. However, in-vitro data on the dissolution of iron pyrophosphate (FePP) in the presence of sodium pyrophosphate (NaPP) suggest that a higher ratio of NaPP:FePP (greater 1 equivalent) further increases iron bioavailability because the presence of multiple pyrophosphate ligands for the iron cation would be available to form soluble complexes.

Patent application WO2016/037836 A1 discloses a water-oil emulsion containing iron(III), and other bivalent cations, for the preparation of margarine or mayonnaise. Example 1, Table 1, reports 1 liter of a suspension of iron pyrophosphate (FePP) in water 8.4 g/liter (37.5 mmoles of Iron(III)) and 1 liter of a solution obtained by adding 10 g of sodium pyrophosphate in 1 liter of water.

Scientific article XP029527322 by Tian Tian et al. discloses that the solubility of iron pyrophosphate depends on pH and the amount of pyrophosphate ions. Tetrasodium pyrophosphate (NaPP) Na4P2O7, molar mass 265.9 g/mol, CAS No. 13472-36-1. Iron pyrophosphate (FePP) Fe4(P2O7)3, molar mass 745.21, CAS No. 10058-44-3.

Patent application WO2014/009806 A1 (Alesco) discloses a composition comprising Iron(III) pyrophosphate from 30% to 70%, sucroester E473 from 10% to 30%, lecithin E322 from 0.1% to 1.5%, where the weight ratio of sucroester:lecithin is from 25:1 to 20:1, and optionally starch from 15% to 40%. Patent application WO2022/190072 A1 (Pharmanutra) discloses a composition comprising Iron(III) pyrophosphate, sucroester, lecithin, where the weight ratio of sucroester:lecithin is from 50:1 to 10:1, and optionally starch.

Patent application EP1743530 A1 discloses, in Examples 3 and 4, the use of gum arabic to prepare Iron(II) pyrophosphate and Iron(III) pyrophosphate nanoparticles.

It is therefore the purpose of the present invention to provide novel oral compositions comprising iron(III) salts that are easily and conveniently formulated and prepared such that they exhibit improved iron absorption and bioavailability, and are thus even more effective.

It is an object of the present invention oral compositions comprising iron (III) salts having the characteristics set forth in the appended claims and their use in therapy or in a method of treatment for prevention or cure.

It is a further object of the present invention oral compositions for use in the preventive and/or curative treatment of anemia or iron deficiency, also in pregnant women and during the postnatal period, having the characteristics set forth in the appended claims.

It is a further object of the present invention a process for the preparation of said oral compositions, as described herein.

### First Aspect of the Invention—Aspect A

According to a first aspect, it is an object of the invention a mixture, such as a physical mixture obtained by mechanical means or by mixing, comprising or, alternatively, consists of at least one iron salt selected from i) an iron (III) pyrophosphate salt, ii) an iron (III)-sodium pyrophosphate salt and/or a mixture thereof, and iii) a sodium or potassium pyrophosphate.

Iron (III) pyrophosphate, iron (III)-sodium pyrophosphate, and sodium or potassium pyrophosphate are all salts or compounds known in the prior art and, at a temperature of 25° C. and pressure of 1 atmosphere, they all occur in solid form, e.g., as a powder or granules.

Preferably, said i) iron (III) pyrophosphate according to the invention is a hydrated salt and, preferably, has a chemical formula, for example, of the type [Fe₄(P₂O₇)3×H₂O] (CAS RN. 10058-44-3, molecular weight of the dry 745.22) and, preferably, may have, for example, an iron content from 15% to 30%, preferably from 18% to 24%, more preferably from 20% to 22% by weight, with respect to the total weight of the molecule. Preferably, said ii) iron (III)-sodium pyrophosphate is a salt and, preferably, has a chemical formula, for example, of the type Fe(III)NaO₇P₂(CAS RN. 10045-87-1).

Preferably, said iii) sodium or potassium pyrophosphate (this description is always intended to include sodium and/or potassium pyrophosphate, even where not explicitly stated) is a salt and, preferably, may be in the form of, for example, a tetrasodium pyrophosphate having a chemical formula, for example, of the type Na₄P₂O₇ (CAS RN. 1269628-79-6) e.g., in anhydrous, semi-hydrated or hydrated form or otherwise with a number of water molecules known to the expert in the field. Tetrasodium pyrophosphate, at room temperature and pressure of 25° C. and 1 atmosphere, occurs as a colorless, odorless, water-soluble solid and is coded in the list of food additives as, for example, E450. Tetrasodium pyrophosphate is normally used in the food industry.

The Applicant surprisingly found that putting in contact in the form of, for example, a combination or association, a sodium pyrophosphate with an iron(III)pyrophosphate and/or an iron(III)-sodium pyrophosphate increases the absorption and bioavailability of iron administered in the body. In particular, the Applicant surprisingly found that a mechanical mixture or a mixture, preferably obtained, for example, by a series of mechanical processing, of a sodium pyrophosphate, e.g., a tetrasodium pyrophosphate, and an iron (III)-pyrophosphate and/or an iron (III)-sodium pyrophosphate, completely unexpectedly, significantly increases iron up-take in the body.

According to an embodiment, it is an object of the invention a mixture AM-1, comprising or alternatively consisting of i) an iron (III)-pyrophosphate salt and iii) a sodium pyrophosphate salt, in a weight ratio iron (III) pyrophosphate:sodium pyrophosphate, e.g., preferably in the form of tetrasodium pyrophosphate, ranging from 1:0.01 to 1:1, preferably in a weight ratio from 1:0.05 to 1:0.9, more preferably from 1:0.1 to 1:0.8, even more preferably from 1:0.15 to 1:0.75, e.g. 1:0.20, or 1:0.25, or 1:0.30, or 1:0.35, or 1:0.40, or 1:0.45, or 1:0.50, or 1:0.55, or 1:0.60, or 1:0.65, or 1:0.70.

According to an embodiment, it is an object of the invention a mixture AM-2, comprising or alternatively consisting of ii) an iron (III)-sodium pyrophosphate salt and iii) a sodium pyrophosphate salt, in a weight ratio iron (III)-sodium pyrophosphate:sodium pyrophosphate, e.g. preferably in the form of tetrasodium pyrophosphate, ranging from 1:0.01 to 1:1, preferably in a weight ratio from 1:0.05 to 1:0.9, more preferably from 1:0.1 to 1:0.8, even more preferably from 1:0.15 to 1:0.75, e.g. 1:0.20, or 1:0.25, or 1:0.30, or 1:0.35, or 1:0.40, or 1:0.45, or 1:0.50, or 1:0.55, or 1:0.60, or 1:0.65, or 1:0.70.

According to an embodiment, it is an object of the invention a mixture AM-3, comprising or alternatively consisting of i) an iron (III)pyrophosphate salt and ii) an iron (III)-sodium pyrophosphate salt, and iii) sodium pyrophosphate, in a weight ratio iron (III)pyrophosphate:iron (III)-sodium pyrophosphate:sodium pyrophosphate, e.g. preferably in the form of tetrasodium pyrophosphate, ranging from 1:0.01:0.01 to 1:0.1:0.1, preferably in a weight ratio ranging from 1:0.05:0.5 to 1:0.5:1:0.5:0.5, more preferably 1:1:1. “Mechanical mixture,” or “mixture, preferably obtained, for example, by a series of mechanical processes”, is understood here to mean that the components or salts of the mixture are all mixed in a solid state, through the use of techniques and equipment known to the expert in the field.

The mixtures AM-1, AM-2 and AM-3 are then prepared by mixing together the components or salts i) and ii) and/or iii) of the mixture in the solid state, preferably in the form of powder or granules.

According to aspect A, it is also an object of the invention a process for preparing mixtures AM-1, AM-2 and AM-3 comprising at least one step of mixing, by the use of mixing means, the components of said mixtures in a solid state, preferably in the form of powder or granules.

It is an object of the present invention a mixture (AM-1, AM-2 or AM-3) comprising or alternatively consisting of:
at least one iron (III) salt selected from i) iron (III) pyrophosphate and ii) iron (III)-sodium pyrophosphate and their mixtures, and
iii) a sodium or potassium pyrophosphate.

Preferably, said mixture consists of i) iron (III) pyrophosphate and iii) sodium or potassium pyrophosphate, preferably said iii) sodium pyrophosphate is tetrasodium pyrophosphate.

Preferably, said i) iron (III) pyrophosphate and iii) sodium or potassium pyrophosphate are present in said mixture in a weight ratio iron (III) pyrophosphate:sodium or potassium pyrophosphate from 1:0.05 to 1:1, preferably from 1:0.1 to 1:0.5, more preferably from 1:0.2 to 1:0.4.

Preferably, said mixture consists of ii) iron (III)-sodium pyrophosphate and iii) sodium or potassium pyrophosphate; preferably said iii) sodium pyrophosphate is tetrasodium pyrophosphate.

Preferably, said ii) iron (III)-sodium pyrophosphate and iii) sodium or potassium pyrophosphate are present in said mixture in a weight ratio iron (III)-sodium pyrophosphate:sodium or potassium pyrophosphate from 1:0.05 to 1:1, preferably from 1:0.1 to 1:0.5, more preferably from 1:0.2 to 1:0.4.

Preferably, said mixture consists of i) iron (III) pyrophosphate, ii) iron (III)-sodium pyrophosphate and iii) sodium or potassium pyrophosphate; preferably said iii) sodium pyrophosphate is tetrasodium pyrophosphate.

Preferably, said mixture is for use in therapy; preferably said mixture is for use in a method of treating and/or preventing conditions of total or relative iron deficiency, particularly for use in the treatment of disorders or diseases related to or resulting from iron deficiency.

It is an object of the present invention a composition (AC-1, AC-2 or AC-3) comprising a mixture (AM-1, AM2 or AM-3) as described above and optionally to at least one excipient and/or vehicle of pharmaceutical or food grade. Preferably, said composition is in solid form in oral dosage units; more preferably, said composition may, in addition, comprise mineral salts and/or vitamins.

According to aspect A, it is also an object of the invention compositions herein referred to as AC-1, AC-2 and AC-3 that comprise a mixture selected from AM-1, AM-2 and AM-3, respectively, together with, optionally, at least one excipient and/or vehicle of pharmaceutical or food grade.

Compositions AC-1, AC-2 and AC-3 are prepared and formulated to be suitable for oral administration.

The compositions AC-1, AC-2 and AC-3 for oral use according to the invention are preferably solid-state compositions formulated in dosage units. Solid state means that the composition can exist in the form of granules or microgranules or powders. The granular or powder compositions are then mixed with pharmacologically acceptable additives and excipients to provide an end product such as, for example, a supplement product, a medical device composition, or a pharmaceutical composition. The final product may be in pharmaceutical dosage units such as granules in sachet, stick, tablet, or capsule.

Tablets can have, for example, different shapes among those known in the field of pharmaceutical forms, such as a cylindrical or spheroidal shape. Tablets can range in weight from 100 mg to 2000 mg. Tablets may be coated or filmed with one or more layers of coating or film capable of passing through the gastric barrier, according to methods and equipment known to the expert in the field.

Gel capsules can have, for example, a weight from 200 mg to 1200 mg, a hard capsule can have a weight from 500 mg to 1000 mg, and a chewable tablet can have a weight from 500 mg to 2000 mg. Capsules may be made of hard gelatin or soft gelatin or soft gel.

Preferably, the oral composition of the invention is a solid composition, as described above. However, if desired or necessary, the composition may also be formulated in liquid form, e.g., as a suspension in water, preferably added with a physiologically acceptable acid, e.g., citric acid, and other substances or excipients capable of keeping the suspension stable and acceptable to the subject taking it.

The oral compositions AC-1, AC-2 and AC-3 of the invention may contain, as mentioned above, physiologically acceptable conventional excipients and vehicles, such as diluents, bulking agents, binders, disaggregants, flow aids, lubricants, etc. Non-limiting examples of suitable vehicles and excipients are described in “Remington: The Science and Practice of Pharmacy, 21st Ed., Lippincott, Williams & Wilkins”.

For example, the compositions of the invention may include cellulose derivatives, glucose, lactose, sucrose, gelatin, malt, rice, flour, gypsum, silica gel, sodium stearate, glycerol mono-stearate, talc, sodium chloride, skim milk powder, glycerol, propylene, glycol, water, ethanol, and the like. The composition may also contain pH buffering reagents and wetting or emulsifying agents, commonly used in the industry.

In addition to the components described above, if desired or necessary, the compositions of the invention may include additional active components, for example, components that can assist the active ingredient in carrying out its action in the organism of the treated subject, such as vitamins.

The oral compositions AC-1, AC-2, and AC-3 according to the invention, in solid or liquid form, are for use in therapy, in particolar for use in therapy to treat conditions of total or relative iron deficiency, particularly for use in the treatment of disorders or diseases related to or resulting from iron deficiency.

The oral compositions AC-1, AC-2, and AC-3 for oral use, solid or liquid, according to any of the embodiments described above, are especially useful in the treatment and prevention of disorders or diseases related to iron deficiency in pediatric age, adolescents, athletes, men, women, pregnant women, and the elderly because they prevent and counteract anemia and are useful in increasing hemoglobin and ferritin values. Said compositions are suitable for administration over a period from 1 month to 6 months, preferably from 2 months to 4 months, in pediatric subjects, adolescents, sportspersons, athletes, men, women, pregnant women, and the elderly, at a dose from 5 mg to 50 mg of iron (III)/day, preferably from 10 mg to 45 mg of iron (III)/day, more preferably from 15 mg to 40 mg, even more preferably from 20 mg to 30 mg, e.g., 25 mg, or 27 mg, or 29 mg, of iron (III)/day.

It is also an object of the invention an iron supplement comprising one of the oral compositions AC-1, AC-2, and AC-3 of the invention, optionally together with other components, for example selected from minerals and/or vitamins, such as at least one vitamin of the group B, C, or D.

It is also an object of the invention a method for the treatment and/or prevention of disorders or diseases related to iron deficiency, comprising administering to a subject in need an oral composition selected from compositions AC-1, AC-2 and AC-3 according to the invention.

The compositions AC-1, AC-2 and AC-3 of the invention comprise iron (III) pyrophosphate salt in an amount by weight from 10% to 90%, preferably from 25% to 75%, more preferably from 35% to 65%, even more preferably from 40% to 55% by weight, with respect to the total weight of the composition.

Preferably, in mixtures AM-1, AM-2 and AM-3 and in compositions AC-1, AC-2 and AC3 of the present invention, said i) an iron (III) pyrophosphate salt is present in said mixtures in an amount by weight from 10% to 90%, more preferably from 25% to 75%, more preferably from 35% to 65%, even more preferably from 40% to 55% by weight, with respect to the total weight of said mixtures.

Preferably, in mixtures AM-1, AM-2 and AM-3 and in compositions AC-1, AC-2 and AC3 of the present invention, said salt i) iron (III) pyrophosphate salt is always present in an amount by weight, with respect to total weight, greater than 10% and less than 75%, by weight.

Preferably, the mixtures AM-1, AM-2 and AM-3 and compositions AC-1, AC-2 and AC3 of the present invention, are both in solid form, e.g., as powders or granules, and are administered in the form of, for example, tablets, capsules, sachets or sticks. The powders and granules, of the mixtures AM-1, AM-2 and AM-3 and the compositions AC-1, AC-2 and AC3 of the present invention, are produced by a process employing equipment and techniques known to the expert in the field, in that the individual components i)-ii)-iii) in the solid state are dry mixed and processed without the use of liquid solutions or water.

Preferably, in mixtures AM-1, AM-2, and AM-3 and compositions AC-1, AC-2, and AC3 of the present invention, iron pyrophosphate is present in a weight ratio greater than 1, with respect to sodium pyrophosphate, preferably tetrasodium pyrophosphate ((FePP:NaPP)>1); or iron pyrophosphate is present in a molar ratio greater than 1, with respect to sodium pyrophosphate, preferably tetrasodium pyrophosphate; or iron pyrophosphate is present in an equivalents ratio greater than 1 ((FePP:NaPP)>1 equivalent)).

Preferably, the weight ratio FePP:NaPP is from greater 1 to 15; more preferably said weight ratio is from 2 to 10; even more preferably said weight ratio is from 3 to 6, still even more preferably said weight ratio may be 3.5; or 4; or 4.5; or 5; or 5.5.

Preferably, the molar or millimolar ratio FePP:NaPP is from greater than 1 to 15; more preferably said molar or millimolar ratio is from 2 to 10; even more preferably said molar or millimolar ratio is from 3 to 6, still even more preferably said molar or millimolar ratio may be 3.5; or 4; or 4.5; or 5; or 5.5.

Preferably, the equivalents ratio FePP:NaPP is from greater 1 to 15; more preferably said equivalents ratio is from 2 to 10; even more preferably said equivalents ratio is from 3 to 6, still even more preferably said equivalents ratio may be 3.5; or 4; or 4.5; or 5; or 5.5.

It is an object of the present invention a process for the preparation of a mixture AM-1, AM-2 and AM-3 and a composition AC-1, AC-2 and AC3, wherein said process comprises at least one step of mixing the individual components i) and/or ii) and/or iii) in the solid state to give said mixture and said composition in the solid state, preferably as powders or granules.

### Second Aspect of the Invention—Aspect B

As mentioned above, many iron-containing compositions present present a difficulty in formulation and show a tendency to give insoluble precipitates that makes their dosing and use difficult. In addition to this, they are often poorly tolerated gastrically and also have very poor palatability.

In order to overcome the above drawbacks, the Applicant has conducted prolonged research that has led to the development of some compositions comprising iron (III) pyrophosphate and the filing of two international patent applications WO2014/009806 and WO2015/033216. These applications (and related granted patents) describe and claim compositions comprising minerals, and in particular iron (III) pyrophosphate, together with sucroesters and lecithins, and are commercially available under the trade name “Sideral©”.

Said compositions provide an improved absorption profile of minerals, and iron in particular, compared to previously used compositions while exhibiting good tolerability and organoleptic stability.

However, the Applicant's intense research activity did not stop with the above compositions but continued with the intention of further improving said compositions, particularly in terms of iron absorption and bioavailability.

Thus, another purpose of the present invention is to provide an oral composition comprising iron (III) salts, which is formulated and prepared in such a way that it exhibits improved absorption and bioavailability of iron and is thus even more effective.

A further purpose of the invention is to provide an oral composition comprising iron (III) salts that is well tolerated by the body, so that it can be administered even on an empty stomach, to all subjects, including pregnant women, that has good palatability and is chemically and organoleptically stable over time, i.e., that does not present changes in color, odor, flavor and taste over time.

In another of its aspects, aspect B1, it is an object of the invention a mixture BM-1 comprising or alternatively consisting of:
i) an iron (III) pyrophosphate salt;
at least one lecithin;
at least one sucroester; and
iii) a sodium pyrophosphate salt, preferably tetrasodium pyrophosphate.

In another of its aspects, aspect B2, it is an object of the invention a mixture BM-2 comprising or alternatively consisting of:
i) an iron (III) pyrophosphate salt;
at least one lecithin;
at least one sucroester;
iii) a sodium pyrophosphate salt, preferably tetrasodium pyrophosphate; and
ii) an iron (III)-sodium pyrophosphate salt.

Preferably, the mixture BM-1 or BM-2 of the present invention may comprise, in addition, an additional component selected from plant starches. Preferably, the plant starch is, for example, preferably selected from rice starches and/or corn starches and/or sunflower starches and/or soybean starches, and mixtures thereof. For example, the preferred starch is a rice starch such as a gelatinized or pregelatinized native rice starch. For example, a pregelatinized rice starch that can be used has a CAS No. 9005-25-8; EINECS 232-679-6 with a moisture content from 10% to 20%, e.g., about 15%; a particle size (particle size distribution) D10 μm max. 20; D50 μm max. 75; and D90 μm max. 175. A commercial product that meets these characteristics is Remyline AX-FG-P from the company A.D.E.A. Srl.

Preferably, another type of pregelatinized rice starch that can be used in mixtures BM-1 or BM-2 can have the following physicochemical characteristics: moisture from 1% to 10%; protein content from 0.1% to 1.5%; ash content from 0.1% to 1%; pH (10% solution) from 5.5 to 7.5; density 0.40-0.48 g/cm³; starch content from a minimum of 95% to 99% and fat content from 0.01% to 0.1%.

Preferably, gelatinized or pregelatinized plant starch is present in mixtures BM-1 and BM-2 in an amount by weight from 1% to 50%, preferably from 10% to 40%, more preferably from 15% to 35%, even more preferably from 20% to 30% by weight, with respect to the total weight of compositions BC-1 and BC-2.

Iron (III) pyrophosphate, iron (III)-sodium pyrophosphate, and sodium pyrophosphate are all known salts or compounds in the prior art.

Preferably, said i) iron (III) pyrophosphate according to the invention is a hydrated salt and, preferably, has a chemical formula, for example, of the type [Fe₄(P₂O₇)3×H₂O] (CAS RN. 10058-44-3, molecular weight of the dry 745.22) and, preferably, may have, for example, an iron content from 15% to 30%, preferably from 18% to 24%, more preferably from 20% to 22% by weight, with respect to the total weight of the molecule.

Preferably, said ii) iron (III)-sodium pyrophosphate is a salt and, preferably, has a chemical formula, for example, of the Fe(III)NaO₇P₂(CAS RN. 10045-87-1).

Preferably, said iii) sodium or potassium pyrophosphate is a salt and, preferably, may be in the form of, for example, a tetrasodium pyrophosphate having a chemical formula, for example, of the type Na₄P₂O₇ (CAS RN. 1269628-79-6). Tetrasodium pyrophosphate, at room temperature and pressure (25° C. and 1 atmosphere), occurs as a colorless, odorless, water-soluble solid and is coded in the list of food additives as E450. Tetrasodium pyrophosphate is normally used in the food industry.

Preferably, said i) iron (III) pyrophosphate salt according to the invention is present in the mixture BM-1 or BM-2 in an amount by weight from 10% to 90%, preferably from 25% to 75%, more preferably from 35% to 65%, even more preferably from 40% to 55% by weight, with respect to the total weight of said mixture.

Preferably, said ii) iron (III)-sodium pyrophosphate salt according to the invention is present in the mixture BM-1 or BM-2in an amount by weight from 0.1% to 50%, preferably from 1% to 35%, more preferably from 5% to 30%, even more preferably from 10% to 25% by weight, with respect to the total weight of said mixture.

Preferably, said iii) sodium or potassium pyrophosphate salt, preferably in the form of tetrasodium phosphate, according to the invention is present in the −2 mixture BM-1 or BM in an amount by weight from 0.1% to 50%, preferably from 1% to 35%, more preferably from 5% to 30%, even more preferably from 10% to 25% by weight, with respect to the total weight of said mixture.

Sodium or potassium pyrophosphate salt, preferably in the form of tetrasodium phosphate, is present in the oral composition BC-1 or BC-2 of the present invention in an amount by weight from 0.1% to 50%, preferably from 1% to 35%, more preferably from 5% to 30%, even more preferably from 10% to 25% by weight, e.g. from 12% to 20%, e.g. 14%, or 16%, or 18% with respect to the total weight of the composition.

The term “lecithin” is known in the technique and is categorized as the food additive, e.g., food additive E322, according to Directive No. 95/2/EC of February 20, 95, published in O.J. No. L61 of September 18, 95.

Lecithin, due to its physicochemical properties, mainly performs an emulsifying function, and in addition, since it is also rich in natural antioxidants, it also has a secondary antioxidant function.

Directive No. 2008/84/EC of Aug. 27, 2008 (published in the O.J. of the European Community No. L253 establishes the purity criteria that lecithin must have in order to be considered food grade (E322): Insoluble in acetone (practically the active part of lecithin): 60% minimum; Moisture: 2% maximum; Acidity number: 35 maximum; Peroxide number: 10 maximum; Insoluble in toluene (practically impurities): 0.3% maximum.

Chemically, it is known that lecithin is a mixture of phosphoric acid, choline, fatty acids, glycerol, glycolipids, triglycerides, and phospholipids.

Phospholipids constitute the main components of lecithins; they are derived from the structure of triglycerides, in which a fatty acid is replaced by a phosphate group, conferring a negative charge, and thus polarity, to the molecule; such a molecule has the generic name phosphatide. A more complex organic molecule, normally serine, choline, ethanolamine, inositol or a single hydrogen atom is linked to the phosphate group through an ester bond, resulting in a phospholipid called, respectively, phosphatidylserine, phosphatidylcholine, phosphatidylethanolamine, phosphatidylinositol or phosphatidic acid. In a narrower sense, phosphatidylcholine is often referred to as lecithin.

Phospholipids are characterized by a water-soluble polar head, which dissolves well in water, while the two saturated fatty acids represent the two apolar, nonwater-soluble but lipophilic tails. These molecules are called amphipathic, and when in the presence of water and fat, they arrange themselves between the fat and water molecules, emulsifying them. Lecithin is therefore a natural emulsifier.

The lecithin used according to the invention is a lecithin as such of those commercially available, including those of allergen-free grade; preferably, it may be a non-hydrolyzed lecithin, such as a lecithin powder.

Preferably, a lecithin selected from lecithins of plant origin such as, for example, sunflower and/or corn and/or soy and/or rice lecithins, and mixtures thereof, may be used.

Preferably, the lecithin used according to the invention is a lecithin powder having, for example, a water content from 0.5% to 10%, preferably from 1.5% to 4.5%, more preferably from 2% to 4%, even more preferably from 2.5% to 3.5%. Preferably, the lecithin used is a sunflower lecithin powder.

Preferably, the sunflower or corn or soybean or rice lecithin or mixtures thereof has an amount by weight of glucose from 20% to 60%, more preferably from 30% to 50%, e.g., about 45% by weight.

Preferably, a sunflower or corn or soybean or rice lecithin or mixtures thereof usable in the context of the present invention may have, for example, the following weight composition (chimica-physical analysis): sunflower or corn or soy lecithin from 20% to 80%, preferably from 40% to 50%, carbohydrates from 30% to 60%, preferably from 40% to 50% (e.g., about 35% or 45% or 55%), protein from 6% to 10%, ash from 3% to 8%, moisture from 2% to 5%, and a flow agent from 0.5% to 1.5%.

Preferably, one type of lecithin usable according to the present invention is a sunflower lecithin of the spray-dried type on glucose syrup (Evra®); this lecithin is sold in the form of a composition comprising glucose syrup, sunflower lecithin, sodium caseinate and tricalcium phosphate. Preferably, another type of lecithin that can be used according to the present invention is a (allergen-free) sunflower lecithin of the spray-dried type on rice flour (Evra®); this lecithin is sold in the form of a composition comprising sunflower lecithin, tricalcium phosphate, and rice flour.

Preferably, the lecithin is present in the oral composition of the present invention in an amount by weight from 0.1% to 10%, preferably from 0.5% to 5%, more preferably from 1% to 4%, even more preferably from 1.5% to 3.5%, e.g. 2%, or 2.5%, or 3% by weight, with respect to the total weight of the composition.

The term “sucroester” according to the invention is known to the art and designates a product obtained by esterification or transesterification of methyl esters of fatty acids with carbohydrates, usually sucrose and other polysaccharides, which is why it is also referred to as “sucrose esters with fatty acids” here also just “sucrose esters.” The chemical and physical properties of these esters depend on the number and type of fatty acids esterified.

Preferably, according to the invention, the at least one sucroester is, for example, a sucroester of the type E473. It is known that the abbreviation E473 indicates that sucroesters are food additives authorized by European Union legislation and regulated by the Italian Ministerial Decree (M.D. 1996). They are essentially emulsifying agents and are added for the purpose of achieving better stabilization between an aqueous and a fatty phase.

Preferably, sucroesters according to the invention are, for example, sucroesters of the type E473 and are used in the composition of the present invention having an HLB value of about 14-18, advantageously an HLB value of about 15 or 16, as emulsifiers, where HLB indicates “Hydrophilic-Lipophilic Balance”.

Preferably, a sucroester for example of the type E473 contains from 50% to 80%, preferably from 60% to 70% of mono-esters, obtained by esterification with fatty acids of plant origin (stearic and palmitic).

Preferably, a type of sucroester usable in the context of the present invention may have, for example, the following composition by weight: a total ester content from 80% to 95%; a free fatty acid content (as oleic acid) from 0.1% to 10%, preferably from 0.5% to 5%, more preferably from 1.5% to 4%, e.g. 2%, or 2.5%, or 3%, or 3.5%; a free sucrose content from 0.5% to 5%; a moisture content from 0.5% to 10%, preferably from 1% to 5%, more preferably from 2% to 4%; an acidity value from 1 mg to 10 mg KOH/g, preferably from 2.5 mg to 5 mg KOH/g. According to the invention, sucrose esters SP70 from the company Sisterna BV, Netherlands, may be used, for example.

Preferably, sucroesters are present in the oral composition of the present invention in an amount by weight from 5% to 75%, preferably from 10% to 60%, more preferably from 12% to 40%, even more preferably from 15% to 30%, e.g., from 16% to 18% by weight, with respect to the total weight of the composition.

It is an object of the present invention a mixture (BM-1 or BM-2) comprising or alternatively consisting of:
i) an iron (III) pyrophosphate salt;
at least one lecithin;
at least one sucroester; and
iii) a sodium or potassium pyrophosphate salt.

Preferably, said mixture also comprises ii) an iron (III)-sodium pyrophosphate salt; preferably said ii) an iron (III)-sodium pyrophosphate salt is present in said mixture in an amount by weight from 0.1% to 50%, preferably from 1% to 35%, more preferably from 5% to 30%, even more preferably from 10% to 25% by weight, with respect to the total weight of said mixture.

Preferably, said mixture also comprises a starch, preferably said starch is a plant starch selected from the group comprising or alternatively consisting of rice starch, corn starch, sunflower starch, or soybean starch, more preferably said plant starch is a pregelatinized rice starch.

Preferably, said starch is present in said mixture in an amount by weight from 1% to 50%, preferably from 10% to 40%, more preferably from 15% to 35%, even more preferably from 20% to 30% by weight, with respect to the total weight of said mixture.

Preferably, said i) an iron (III) pyrophosphate salt is present in said mixture in an amount by weight from 10% to 90%, preferably from 25% to 75%, more preferably from 35% to 65%, even more preferably from 40% to 55% by weight, with respect to the total weight of said mixture.

Preferably, said at least one lecithin is a plant lecithin, preferably said plant lecithin is selected from the group comprising or alternatively consisting of a sunflower, corn, soy, or rice lecithin; preferably said at least one lecithin is present in said mixture in an amount by weight from 0.1% to 10%, preferably from 0.5% to 5%, more preferably from 1% to 4%, even more preferably from 1.5% to 3.5% by weight, with respect to the total weight of said mixture.

Preferably, said at least one sucroester is present in said mixture in an amount by weight from 5% to 75%, preferably from 10% to 60%, more preferably from 12% to 40%, even more preferably from 15% to 30% by weight, with respect to the total weight of said mixture.

Preferably, said iii) sodium or potassium pyrophosphate salt, preferably in the form of tetrasodium pyrophosphate, is present in said mixture in an amount by weight from 0.1% to 50%, preferably from 1% to 35%, more preferably from 5% to 30%, even more preferably from 10% to 25% by weight, with respect to the total weight of said mixture.

Preferably, said mixture is for use in therapy; preferably said mixture is for use in a method of treatment and/or prevention of conditions of total or relative iron deficiency, particularly for use in the treatment of disorders or diseases related to or resulting from iron deficiency.

It is an object of the present invention a composition (BC-1 or BC-2) comprising a mixture (BM-1 or BM-2) as described above and, optionally, at least one excipient and/or vehicle of pharmaceutical or food grade; preferably said composition is in solid form in oral dosage units.

It is also an object of the invention a composition BC-1 composition comprising or, alternatively, consisting of a mixture BM-1, together with at least one excipient and/or vehicle of pharmaceutical or food grade.

It is also an object of the invention a composition BC-2 composition comprising or, alternatively, consisting of a mixture BM-2, together with at least one excipient and/or vehicle of pharmaceutical or food grade.

Compositions BC-1 and BC-2 are prepared to be suitable for oral administration.

The compositions BC-1 and BC-2 according to the invention are preferably a solid state composition formulated in dosage units. Solid state means that the composition can exist in the form of granules or powders. The granular or powder compositions are then mixed with pharmacologically acceptable additives and excipients to provide an end product such as a supplement product, medical device composition, or pharmaceutical composition. The final product may be in pharmaceutical dosage units such as granules in sachets, or sticks, tablets, or capsules.

For example, tablets can have different shapes among those known in the field of pharmaceutical forms, such as a cylindrical or spheroidal shape. Tablets can have, for example, a weight from 100 mg to 2000 mg. Tablets may be coated or filmed with one or more layers of coating or film capable of passing through the gastric barrier, according to methods and equipment known to the expert in the field.

For example, gel capsules may have a weight of 200-1200 mg, a hard capsule may have a weight from 500 mg to 1000 mg, and a chewable tablet may have a weight from 500 mg to 2000 mg. Capsules may be made of hard gelatin or soft gelatin or soft gel.

Preferably, the oral composition of the invention is a solid composition, as described above. However, if desired or necessary, the composition may also be formulated in liquid form, e.g., as a suspension in water, preferably added with a physiologically acceptable acid, e.g., citric acid, and other substances or excipients capable of keeping the suspension stable and acceptable to the subject taking it.

Solid compositions of the invention may contain, as mentioned above, physiologically acceptable conventional excipients and vehicles of pharmaceutical or food grade, such as diluents, bulking agents, binders, disaggregants, flow aids, lubricants, etc. Non-limiting examples of suitable vehicles and excipients are described in “Remington: The Science and Practice of Pharmacy, 21st Ed., Lippincott, Williams & Wilkins”. For example, compositions of the invention may also include cellulose derivatives, glucose, lactose, sucrose, gelatin, malt, rice, flour, gypsum, silica gel, sodium stearate, glycerol mono-stearate, talc, sodium chloride, skim milk powder, glycerol, propylene, glycol, water, ethanol, and the like. The composition may also contain, for example, pH buffering reagents and wetting or emulsifying agents.

In addition to the components described above, if desired or necessary, the compositions BC-1 and BC-2 of the invention may also preferably comprise additional active components, for example, components that can assist the active ingredient in carrying out its action in the organism of the treated subject, such as vitamins.

The oral compositions BC-1 and BC-2 according to the invention are for use in therapy, particularly in conditions of total or relative iron deficiency, especially for use in the treatment of disorders or diseases related to or resulting from iron deficiency.

The composition for oral use, solid or liquid, according to any of the embodiments described above, is useful in therapy, particularly in a method of treatment and prevention of disorders or diseases related to iron deficiency in pediatric subjects, adolescents, athletes, men, women, pregnant women, and the elderly since it prevents and counteracts anemia and is useful in increasing hemoglobin and ferritin values. Said composition is suitable for administration over a period from 1 month to 12 months, preferably from 2 months to 6 months, in pediatric subjects, adolescents, sportspersons, athletes, men, women, pregnant women, and the elderly, at a dose, for example, from 5 mg to 50 mg of iron (III)/day, preferably from 10 mg to 45 mg of iron (III)/day, more preferably from 15 mg to 40 mg, even more preferably from 20 mg to 30 mg, e.g., 25 mg, or 27 mg, or 29 mg, of iron (III)/day.

It is also an object of the invention an iron supplement comprising a composition BC-1 or BC-2 of the invention, possibly together with other components, for example selected from minerals and/or vitamins such as, for example, one or more vitamins belonging to the B, C and D groups.

It is also an object of the invention a method for the treatment and/or prevention of disorders or diseases related to iron deficiency, comprising administering a composition BC-1 or BC-2 according to the invention to a subject in need.

The compositions BC-1 and BC-2 of the invention comprise iron (III) pyrophosphate salt in an amount by weight from 10% to 90%, preferably from 25% to 75%, more preferably from 35% to 65%, even more preferably from 40% to 55% by weight, with respect to the total weight of the composition.

The Applicant surprisingly found that putting in contact in the form of, for example, a combination or association, a sodium or potassium pyrophosphate salt, preferably a tetrasodium pyrophosphate, with a composition described in WO2014/009806 and WO2015/033216 and marketed under the trade name “Sideral*” increases the absorption and bioavailability of iron administered in the body. Specifically, the Applicant surprisingly found that a mechanical mixture or a mixture, preferably obtained for example by mechanical means, of a sodium or potassium pyrophosphate salt, preferably a tetrasodium pyrophosphate, with a composition described in WO2014/009806 and WO2015/033216, completely unexpectedly, significantly increases iron up-take, preferably through the increase of ferritin.

This surprising result will be described and demonstrated in detail in the Experimental Section that follows.

Preferably, said iii) a sodium or potassium pyrophosphate salt, preferably in the form of tetrasodium pyrophosphate, is present in the oral composition BC-1 or BC-2 of the present invention in an amount by weight from 0.1% to 50%, preferably from 1% to 35%, more preferably from 5% to 30%, even more preferably from 10% to 25% by weight, e.g. from 12% to 20%, e.g. 14%, or 16%, or 18% with respect to the total weight of the composition.

Preferably, said ii) an iron (III)-sodium pyrophosphate salt is present in the oral composition BC-1 or BC-2 of the present invention in an amount by weight from 0.1% to 50%, preferably from 1% to 35%, more preferably from 5% to 30%, even more preferably from 10% to 25% by weight, e.g. from 12% to 20%, e.g. 14%, or 16%, or 18% with respect to the total weight of the composition.

The oral composition BC-1 object of the present invention therefore comprises or alternatively consists of iron (III) pyrophosphate, a lecithin, e.g., sunflower lecithin, sucrose esters or sucroesters, e.g., E473, and a sodium or potassium salt, preferably in the form of tetrasodium pyrophosphate, in the above percent amounts by weight.

The oral composition BC-2 object of the present invention therefore comprises or alternatively consists of iron (III) pyrophosphate, a lecithin, e.g., sunflower lecithin, sucrose esters or sucroesters, e.g., E473, and a sodium or potassium salt, preferably in the form of tetrasodium pyrophosphate, in the above percent amounts by weight.

As mentioned above, the oral composition BC-1 or BC-2 of the present invention may also comprise other conventional, physiologically acceptable, components, excipients, and vehicles.

Preferably, the oral composition BC-1 or BC-2 of the present invention may comprise, in addition, an additional component selected from plant starches. Preferably, the plant starch is, for example, preferably selected from rice starches or corn starches or sunflower starches. For example, the preferred starch is a rice starch such as a gelatinized or pregelatinized native rice starch. For example, a pregelatinized rice starch that can be used has a CAS No. 9005-25-8; EINECS 232-679-6 with a moisture content from 10% to 20%, e.g., about 15%; a particle size (particle size distribution) D10 μm max. 20; D50 μm max. 75; and D90 μm max. 175. A commercial product that meets these characteristics is Remyline AX-FG-P from the company A.D.E.A. Srl.

Preferably, another type of pregelatinized rice starch that can be used within the scope of the present invention may have the following physicochemical characteristics: moisture content from 1% to 10%; protein content from 0.1% to 1.5%; ash content from 0.1% to 1%; pH (10% solution) from 5.5 to 7.5; density 0.40-0.48 g/cm³; starch content from a minimum of 95% to 99% and fat content from 0.01% to 0.1%.

Preferably, gelatinized or pregelatinized plant starch is present in the solid composition BC-1 or BC-2 in an amount by weight from 1% to 50%, preferably from 10% to 40%, more preferably from 15% to 35%, even more preferably from 20% to 30% by weight, with respect to the total weight of compositions BC-1 and BC-2.

The oral composition BC-1 object of the present invention comprises or alternatively consists of iron (III) salts, a lecithin, e.g., sunflower lecithin, sucrose esters or sucroester, e.g., E473, a starch, preferably a plant starch such as, for example, rice starch, and a sodium or potassium pyrophosphate salt, preferably in the form of tetrasodium pyrophosphate, in the above percent amounts by weight.

The oral composition BC-1 object of the present invention comprises or alternatively consists of an iron (III) pyrophosphate salt, a lecithin, e.g., sunflower lecithin, sucrose esters or sucroester, e.g., E473, a starch, preferably a plant starch such as, for example, rice starch, and a sodium or potassium pyrophosphate salt, preferably in the form of tetrasodium pyrophosphate, in the above percent amounts by weight.

The oral composition BC-2 object of the present invention comprises or alternatively consists of iron (III) salts, a lecithin, e.g., a sunflower lecithin, sucrose esters or sucroester, e.g., E473, a starch preferably a plant starch, such as, for example, a rice starch, and a sodium or potassium pyrophosphate salt, preferably in the form of tetrasodium pyrophosphate and an iron (III)-sodium pyrophosphate, in the percent amounts by weight indicated above.

The oral composition BC-2 object of the present invention comprises or alternatively consists of an iron (III) pyrophosphate salt, a lecithin, e.g., sunflower lecithin, sucrose esters or sucroester, e.g., E473, a starch preferably a plant starch, such as, for example, a rice starch, and a sodium or potassium pyrophosphate salt, preferably in the form of tetrasodium pyrophosphate and iron (III)-sodium pyrophosphate in the percent amounts by weight indicated above.

It also an object of the present invention a first method for preparing a mixture BM-1 or BM-2 which, when prepared, is added with physiologically acceptable excipients, diluents or carriers to give the oral composition BC-1 or BC-2 of the present invention, respectively.

A first method of the present invention is directed to the preparation of a mixture comprising or, alternatively, consisting of i) iron (III) pyrophosphate, a lecithin, e.g. a sunflower lecithin, sucrose esters or sucroester, e.g. E473, a iii) sodium or potassium pyrophosphate salt, preferably tetrasodium pyrophosphate, optionally a plant starch, preferably a rice starch, to give the mixture BM-1. If an ii) iron (III)-sodium pyrophosphate is, optionally, also added to this mixture BM-1, in the above percent amounts by weight, mixture BM-2 is obtained.

Preferably, said i) iron (III) pyrophosphate in a solid state is, for example, put in contact with a iii) sodium or potassium pyrophosphate salt, preferably in the form of tetrasodium pyrophosphate, and/or ii) iron (III)-sodium pyrophosphate, in the above-mentioned percent amounts by weight, by mixing to give a resulting mixture to which a plant lecithin, optionally a starch, and/or a sucroester are subsequently added.

Preferably, mixing is conducted in a mixer equipped with stirring and mixing means known in the art.

Preferably, sieving steps are provided, using suitable sieves or sieving machine, in order to make the solid powder mixture being processed more uniform and consistent. Some further details of said first method of the invention are given in the Experimental Section of the present invention.

Said i) iron (III) pyrophosphate, lecithin, sucroester, iii) sodium or potassium pyrophosphate (e.g., tetrasodium pyrophosphate), starch, and possibly ii) iron (III)-sodium pyrophosphate used in the preparation method of the present invention have the characteristics and properties defined above.

Plant starch, e.g., in the form of gelatinized or pregelatinized plant starch, is fluid and free-flowing, allowing it to be accurately dosed without causing errors or weight variations. It also distributes more evenly and homogeneously within the mixture during the mixing step. Finally, plant starch improves the bioavailability of the iron cation, as the resulting compound dissolves better at temperatures ranging from 15 to 30° C. (pressure 1 atmosphere), preferably from 20 to 25° C., even more preferably from 18 to 23° C.

Upon completion of said first method of preparation, a mixture BM-1 (and an oral solid composition BC-1) of the present invention is obtained, comprising or, alternatively, consisting of i) an iron (III)-pyrophosphate, a plant lecithin, sucrose esters or sucroester, a iii) sodium or potassium pyrophosphate salt, such as tetrasodium pyrophosphate, and optionally a starch, and possibly a ii) iron (III)-sodium pyrophosphate in the mixture BM-2 (and in the composition BC-2) in the above percent amounts by weight.

Preferably, by said first method of preparation, a mixture BM-1 (and an oral solid composition BC-1) of the present invention is obtained, which comprises or, alternatively, consists of i) an iron (III) pyrophosphate, a plant lecithin, e.g., a corn or soybean or sunflower lecithin E322, sucrose esters or sucroester, e.g., of the type E473, iii) a sodium or potassium pyrophosphate salt (e.g., a tetrasodium pyrophosphate), and, optionally, a starch (e.g., a rice starch), in the above percent amounts by weight.

Preferably, by of said first method of preparation, a mixture BM-2 (and an oral solid composition BC-2) of the present invention is obtained, which comprises or, alternatively, consists of i) an iron (III) pyrophosphate, a plant lecithin, e.g., a corn or soybean or sunflower lecithin E322, sucrose esters or sucroester, e.g., E473, iii) a sodium or potassium pyrophosphate salt (e.g., a tetrasodium pyrophosphate) ii) an iron (III)-sodium pyrophosphate, and optionally, a starch (e.g., a rice starch) in the above percent amounts by weight.

Preferably, to further improve the bioavailability of the iron cation, it is useful to reduce the amount by weight of lecithin to be used in the process to prepare the solid composition of the present invention as much as possible.

In addition, it has been found that to further enhance the bioavailability of the iron cation, it is preferable to use a specific amount by weight of sucrose esters or sucroester in combination with a reduced amount by weight of lecithin.

Preferably, the weight ratio of sucrose ester or sucroester to lecithin is in the range from 60:1 to 10:1, preferably from 50:1 to 20:1, more preferably from 45:1 to 30:1, e.g., from 40:1 to 35:1. In an embodiment, said ratio is in the range from 45:1 to 35:1.

Preferably, lecithin is present in the mixture BM-1 or BM-2 in an amount by weight from 0.01% to 10%, preferably from 0.05% to 5%, more preferably from 0.1% to 3.5%, even more preferably from 0.5% to 2%, e.g., from 0.8% to 1.5%, e.g., 0.9%, or 1%, or 1.1%, or 1.2%, or 1.3%, or 1.4% by weight, with respect to the weight of the mixture.

Said method of the present invention enables the creation of a coating or encapsulation around iron (III) so as to improve the stability and bioavailability of the cation (III) due to the presence of the sodium or potassium pyrophosphate salt and/or iron (III)-sodium pyrophosphate salt.

In practice, said method involves the formation of agglomerates or granules comprising the iron (III) pyrophosphate and the sodium or potassium pyrophosphate salt and/or iron (III)-sodium pyrophosphate salt in the presence of lecithin, sucrose esters or sucroesters, and optionally a starch.

Sucrose esters or sucroesters and lecithin act by promoting the absorption of the salt and, consequently, of the iron cation contained in said salt. The mixture with lecithin and starch results in the formation of “chimeric” agglomerates capable of protecting and shielding from gastric acid the iron cation contained in the pyrophosphate salt.

Processing time, e.g., mixing and sieving, is from 5 to 90 minutes, preferably from 10 to 60, more preferably from 20 to 40 minutes.

The oral composition obtained by the method of the present invention can have a particle size distribution of type D10 of about 1.8 μm, D50 of about 20.5 μm and D90 of about 108 μm.

The solid composition of the present invention has an iron (III) content ranging from 30 mg/g to 180 mg/g, preferably from 60 mg/g to 120 mg/g, more preferably from 90 and 110 mg/g.

The oral composition BC-1 or BC-2 according to the invention is for use in therapy, particularly in conditions of total or relative iron deficiency, especially for use in the treatment of disorders or diseases related to or resulting from iron deficiency.

The oral composition BC-1 or BC-2 of the present invention comprises iron that is easily absorbed and effectively bioavailable, and able to increase ferritin. In addition, the oral composition BC-1 or BC-2 has been shown to be well tolerated by the body. The composition can be administered, even on an empty stomach, to all categories of patients, including pregnant women. Oral composition BC-1 or BC-2 exhibits good palatability and has been shown to be stable over time chemically and organoleptically i.e., no changes in color, odor, taste and/or flavor have been observed.

The compositions BC-1 and BC-2 according to the invention are a solid-state composition formulated in dosage units. Solid state means that the composition can exist in the form of granules, microgranules or powders or flakes. The granular or microgranular or powder compositions are then mixed with pharmaceutically acceptable additives and excipients of pharmaceutical or food grade to provide an end product such as a supplement product, a medical device reg. EU 745/2017 composition, or a pharmaceutical composition. The final product can be in pharmaceutical dosage units such as, for example, granulated sachet, tablet, or capsule.

Tablets can have different shapes among those known in the field of pharmaceutical forms, such as a cylindrical or spheroidal shape. Tablets can range in weight from 100 to 2000 mg. Tablets may be coated or filmed with one or more layers of coating or film capable of passing through the gastric barrier, according to known methods.

Gel capsules may have, for example, a weight from 200 mg to 1200 mg, a hard capsule may have a weight from 500 mg to 1000 mg, and a chewable tablet may have a weight from 500 mg to 2000 mg. Capsules may be made of hard gelatin or soft gelatin or soft gel.

Each dosage unit is from 5 mg to 50 mg of iron(III)/day, preferably from 10 mg to 45 mg of iron(III)/day, more preferably from 15 mg to 40 mg, even more preferably from 20 mg to 30 mg, e.g., 25 mg, or 27 mg, or 29 mg, of iron(III)/day.

As mentioned, preferably the oral composition BC-1 or BC-2 of the invention is a solid composition, as described above. However, if desired or necessary, the composition may be formulated in liquid form, such as suspension in water, preferably added with a physiologically acceptable acid, e.g., citric acid.

The solid compositions of the invention may contain, as mentioned, physiologically acceptable conventional excipients and vehicles, such as diluents, bulking agents, binders, disaggregants, flow aids, lubricants, etc. Non-limiting examples of suitable vehicles and excipients are described in “Remington: The Science and Practice of Pharmacy, 21st Ed., Lippincott, Williams & Wilkins”. The compositions of the invention may, for example, contain cellulose derivatives, glucose, lactose, sucrose, gelatin, malt, rice, flour, gypsum, silica gel, sodium stearate, glycerol monostearate, talc, sodium chloride, skim milk powder, glycerol, propylene, glycol, water, ethanol, and the like. The composition may also contain pH buffering reagents and wetting or emulsifying agents.

Preferred excipients include hydroxypropyl methyl cellulose and magnesium salts of fatty acids.

In addition to the components described above, if desired or necessary, the compositions BC-1 and BC-2 of the invention may include additional active components, such as components that can assist the active ingredient in carrying out its action in the organism of the treated subject, such as vitamins.

It is also an object of the invention a liquid oral composition comprising or alternatively consisting of a composition BC-1 or BC-2 according to the invention, water and citric acid.

The oral composition BC-1 or BC-2, solid or liquid, according to any one of the above-described embodiments, is useful in therapy, particularly in the treatment and prevention of disorders or diseases related to iron deficiency in pediatric subjects, adolescents, athletes, men, women, pregnant women, and the elderly since it prevents and counteracts anemia and is useful in increasing hemoglobin and ferritin values. Said composition BC-1 or BC-2 is suitable for administration over a period from 1 to 5 months, preferably from 2 to 4 months, in pediatric subjects, adolescents, athletes, men, women, pregnant women, and the elderly, at a dose from 10 to 40 mg iron(III)/day, preferably from 14 to 30 mg iron(III)/day, even more preferably 28 mg iron(III)/day.

The compositions BC-1 and BC-2 can be administered throughout the pregnancy period, particularly from the 12th weeks, up to 6 weeks postpartum. The recommended dose of iron (III) is from 10 to 40 mg/day, preferably from 14 to 30 mg/day, advantageously 28 mg/day.

Preferably, in the mixtures BM-1 and BM-2 and in the compositions BC-1 and BC-2 of the present invention, said i) an iron (III) pyrophosphate salt is present in said mixtures in an amount by weight from 10% to 90%, more preferably from 25% to 75%, more preferably from 35% to 65%, even more preferably from 40% to 55% by weight, with respect to the total weight of said mixtures.

Preferably, in the mixtures BM-1 and BM-2 and in the compositions BC-1 and BC-2 of the present invention, said salt i) iron (III) pyrophosphate salt is always present in an amount by weight, with respect to total weight, greater than 10% and less than 75%, by weight.

Preferably, the mixtures BM-1 and BM-2 and in compositions BC-1 and BC-2 of the present invention, are both in solid form, for example, in the form of powders or granules, and are administered in the form of, for example, tablets, capsules, sachets, or sticks. The powders and granules, of mixtures BM-1 and BM-2 and in compositions BC-1 and BC-2 of the present invention, are produced by a process employing equipment and techniques known to the person skilled in the art, wherein the individual components i)-ii)-iii) in the solid state are mixed and processed dry without the use of liquid solutions or water.

Preferably, in the mixtures BM-1 and BM-2 and in the compositions BC-1 and BC-2 of the present invention, iron pyrophosphate is present in a molar ratio greater than 1, with respect to sodium pyrophosphate, preferably tetrasodium pyrophosphate ((FePP:NaPP)>1); or iron pyrophosphate is present in a molar ratio greater than 1, with respect to sodium pyrophosphate, preferably tetrasodium pyrophosphate; or iron pyrophosphate is present in an equivalents ratio greater than 1 ((FePP:NaPP)>1 equivalent)).

Preferably, the weight ratio FePP:NaPP is from greater 1 to 15; more preferably said weight ratio is from 2 to 10; even more preferably said weight ratio is from 3 to 6, still even more preferably said weight ratio may be 3.5; or 4; or 4.5; or 5; or 5.5.

Preferably, the molar or millimolar ratio FePP:NaPP is from greater than 1 to 15; more preferably said molar or millimolar ratio is from 2 to 10; even more preferably said molar or millimolar ratio is from 3 to 6, still even more preferably said molar or millimolar ratio may be 3.5; or 4; or 4.5; or 5; or 5.5. Preferably, the equivalents ratio FePP:NaPP is from greater 1 to 15; more preferably said equivalents ratio is from 2 to 10; even more preferably said equivalents ratio is from 3 to 6, still even more preferably said equivalents ratio may be 3.5; or 4; or 4.5; or 5; or 5.5.

It is an object of the present invention a process for the preparation of a mixture BM-1 and BM-2 and a composition BC-1 and BC-2 wherein said process comprises at least one step of mixing the individual components i) and/or ii) and/or iii) in the solid state to give said mixture and said composition in the solid state, preferably as powders or granules.

### Third Aspect of Invention—Aspect C

In another of its aspects, aspect C1, it is an object of the invention a mixture CM-1 consisting of:
i) an iron (III) pyrophosphate salt;
at least one lecithin;
at least one gum arabic (or acacia gum); and
iii) a sodium or potassium pyrophosphate salt, preferably tetrasodium pyrophosphate.

In another of its aspects, aspect C2, it is an object of the invention a mixture CM-2 consisting of:
i) an iron (III) pyrophosphate salt;
at least one lecithin;
at least one gum arabic (or acacia gum);
iii) a sodium pyrophosphate salt, preferably tetrasodium pyrophosphate; and
ii) an iron (III)-sodium pyrophosphate salt.

Preferably, the mixture CM-1 or CM-2 of the present invention may comprise, in addition, an additional component selected from plant starches. Preferably, the plant starch is, for example, preferably selected from rice starches or corn starches or sunflower starches or soybean starches, or mixtures thereof. For example, the preferred starch is a rice starch such as a gelatinized or pregelatinized native rice starch. For example, a pregelatinized rice starch that can be used has a CAS No. 9005-25-8; EINECS 232-679-6 with a moisture content from 10% to 20%, e.g., about 15%; a particle size (particle size distribution) D10 μm max. 20; D50 μm max. 75; and D90 μm max. 175. A commercial product that meets these characteristics is Remyline AX-FG-P from the company A.D.E.A. Srl.

Preferably, another type of pregelatinized rice starch that can be used in mixtures CM-1 or CM-2 can have the following physicochemical characteristics: moisture from 1% to 10%; protein content from 0.1% to 1.5%; ash content from 0.1% to 1%; pH (10% solution) from 5.5 to 7.5; density 0.40-0.48 g/cm³; starch content from a minimum of 95% to 99% and fat content from 0.01% to 0.1%.

Preferably, gelatinized or pregelatinized plant starch is present in mixtures CM-1 and CM-2 in an amount by weight from 1% to 50%, preferably from 10% to 40%, more preferably from 15% to 35%, even more preferably from 20% to 30% by weight, with respect to the total weight of the compositions CC-1 and CC-2.

Iron (III) pyrophosphate, iron (III)-sodium pyrophosphate, and sodium pyrophosphate are all known salts or compounds in the prior art.

Preferably, said gum arabic or acacia gum is present in said mixture CM-1 or CM-2 in an amount by weight ranging from 5% to 45%, preferably from 10% to 40%, more preferably from 15% to 35%, even more preferably from 20% to 30% by weight, with respect to the total weight of said mixture.

Iron (III) pyrophosphate, iron (III)-sodium pyrophosphate, and sodium pyrophosphate are all known salts or compounds in the prior art and, at a temperature of 25° C., all occur in solid form, e.g., as a powder or granules.

Preferably, said i) iron (III) pyrophosphate according to the invention is a hydrated salt and, preferably, has a chemical formula, for example, of the type [Fe₄(P₂O₇)3×H₂O] (CAS RN. 10058-44-3, molecular weight of the dry 745.22) and, preferably, may have an iron content from 18% to 24%, even more preferably from 20% to 22% by weight, with respect to the total weight of the molecule.

Preferably, said ii) iron (III)-sodium pyrophosphate is a salt and, preferably, has a chemical formula, for example, of the type Fe(III)NaO₇P₂(CAS RN. 10045-87-1).

Preferably, said iii) sodium pyrophosphate is a salt and, preferably, may be in the form of, for example, a tetrasodium pyrophosphate having a chemical formula, for example, of the type Na₄P₂O₇ (CAS RN. 1269628-79-6) e.g., in anhydrous, semi-hydrated or hydrated form or otherwise with a number of water molecules known to the expert in the field. Tetrasodium pyrophosphate, at room temperature and pressure of 25° C. and 1 atmosphere, occurs as a colorless, odorless, water-soluble solid and is coded in the list of food additives as E450. Tetrasodium pyrophosphate is normally used in the food industry.

Preferably, said i) iron (III) pyrophosphate salt according to the invention is present in the mixture CM-1 or CM-2 in an amount by weight from 10% to 90%, preferably from 25% to 75%, more preferably from 35% to 65%, even more preferably from 40% to 55% by weight, with respect to the total weight of said mixture.

Preferably, said ii) iron (III)-sodium pyrophosphate salt according to the invention is present in the mixture CM-1 or CM-2in an amount by weight from 0.1% to 50%, preferably from 1% to 35%, more preferably from 5% to 30%, even more preferably from 10% to 25% by weight, with respect to the total weight of said mixture.

Preferably, said iii) sodium salt or potassium pyrophosphate, preferably in the form of tetrasodium phosphate, according to the invention is present in the mixture CM-1 or CM-2 in an amount by weight from 0.1% to 50%, preferably from 1% to 35%, more preferably from 5% to 30%, even more preferably from 10% to 25% by weight, with respect to the total weight of said mixture.

The term “lecithin” is known to the technique and is categorized as the food additive E322 according to Directive No. 95/2/EC of February 20, 95, published in O.J. No. L61 of September 18, 95.

Lecithin, due to its physicochemical properties, mainly performs an emulsifying function, and in addition, since it is also rich in natural antioxidants, it also has a secondary antioxidant function.

Directive No. 2008/84/EC of Aug. 27, 2008 (published in the O.J. of the European Community No. L253 establishes the purity criteria that lecithin must have in order to be considered food grade (E322): Insoluble in acetone (practically the active part of lecithin): 60% minimum; Moisture: 2% maximum; Acidity number: 35 maximum; Peroxide number: 10 maximum; Insoluble in toluene (practically impurities): 0.3% maximum.

Chemically, lecithin is a mixture of phosphoric acid, choline, fatty acids, glycerol, glycolipids, triglycerides, and phospholipids.

Phospholipids constitute the main components of lecithins; they are derived from the structure of triglycerides, in which a fatty acid is replaced by a phosphate group, conferring a negative charge, and thus polarity, to the molecule; such a molecule has the generic name phosphatide. A more complex organic molecule, normally serine, choline, ethanolamine, inositol or a single hydrogen atom is linked to the phosphate group through an ester bond, resulting in a phospholipid called, respectively, phosphatidylserine, phosphatidylcholine, phosphatidylethanolamine, phosphatidylinositol or phosphatidic acid. In a narrower sense, phosphatidylcholine is often referred to as lecithin.

Phospholipids are characterized by a water-soluble polar head, which dissolves well in water, while the two saturated fatty acids represent the two apolar, nonwater-soluble but lipophilic tails. These molecules are called amphipathic, and when in the presence of water and fat, they arrange themselves between the fat and water molecules, emulsifying them. Lecithin is therefore a natural emulsifier.

The lecithin used according to the invention is an unhydrolyzed, powdered lecithin, and is preferably selected from sunflower and/or corn and/or soybean and/or corn and/or rice lecithin, or mixtures thereof.

The lecithin used according to the invention is a powdered lecithin having a water content from 1.5% to 4.5%, preferably from 2 to 4%, even more preferably from 2.5% to 3.5%. Preferably, the lecithin used is a sunflower lecithin powder.

In an embodiment, sunflower lecithin has an amount of glucose from 20% to 60%, preferably from 30% to 50%, for example, about 45% by weight.

A sunflower lecithin that can be used in the context of the present invention may have the following composition by weight (physicochemical analysis): sunflower lecithin from 40% to 50%, carbohydrates from 40% to 50% (e.g., about 42%), protein from 6% to 10%, ash from 3% to 8%, moisture from 2% to 5%, and a flowing agent from 0.5% to 1.5%.

Lecithin is present in the oral composition of the present invention in an amount by weight from 0.1% to 10%, preferably from 0.5% to 5%, more preferably from 1% to 4%, even more preferably from 1.5% to 3.5% by weight, with respect to the total weight of the composition.

The term “gum arabic” is known to the technique and designates a natural gum also known as acacia gum because it is extracted from two species of acacia, Acacia senegal and Acacia seyal.

Gum Arabic is commercially available and, in the context of the present invention, a gum derived from Acacia senegal of type E414, for example, can be used.

Preferably, a gum arabic (acacia gum) of type E414 can be used, for example, which occurs as a white powder and has a CAS No. 9000-01-5 and EINECS No. 232-519-5, an average molecular weight of about 350000, a viscosity of about 60-130 mPas (sol. 25%) and is practically insoluble in ethanol (96%). It is an object of the present invention a mixture (CM-1 or CM-2) comprising or alternatively consisting of:
i) an iron (III) pyrophosphate salt;
at least one lecithin;
at least one gum arabic or acacia gum; and
iii) a sodium or potassium pyrophosphate salt.

Preferably, said mixture also comprises ii) an iron (III)-sodium pyrophosphate salt; preferably said ii) an iron (III)-sodium pyrophosphate salt is present in said mixture in an amount by weight from 0.1% to 50%, preferably from 1% to 35%, more preferably from 5% to 30%, even more preferably from 10% to 25% by weight, with respect to the total weight of said mixture.

Preferably, said mixture also comprises a starch, preferably said starch is a plant starch selected from the group comprising or alternatively consisting of rice starch, corn starch, sunflower starch, or soybean starch, more preferably said plant starch is a pregelatinized rice starch.

Preferably, said starch is present in said mixture in an amount by weight from 1% to 50%, preferably from 10% to 40%, more preferably from 15% to 35%, even more preferably from 20% to 30% by weight, with respect to the total weight of said mixture.

Preferably, said i) an iron (III) pyrophosphate salt is present in said mixture in an amount by weight from 10% to 90%, preferably from 25% to 75%, more preferably from 35% to 65%, even more preferably from 40% to 55% by weight, with respect to the total weight of said mixture.

Preferably, said at least one lecithin is a plant lecithin, preferably said plant lecithin is selected from the group comprising or alternatively consisting of a sunflower, corn, soy, or rice lecithin; preferably said at least one lecithin is present in said mixture in an amount by weight from 0.1% to 10%, preferably from 0.5% to 5%, more preferably from 1% to 4%, even more preferably from 1.5% to 3.5% by weight, with respect to the total weight of said mixture.

Preferably, said gum arabic or acacia gum is present in said mixture in an amount by weight ranging from 5% to 45%, preferably from 10% to 40%, more preferably from 15% to 35%, even more preferably from 20% to 30% by weight, with respect to the total weight of said mixture.

Preferably, said iii) sodium or potassium pyrophosphate salt, preferably in the form of tetrasodium pyrophosphate, is present in said mixture in an amount by weight from 0.1% to 50%, preferably from 1% to 35%, more preferably from 5% to 30%, even more preferably from 10% to 25% by weight, with respect to the total weight of said mixture.

Preferably, said mixture is for use in therapy; preferably said mixture is for use in a method of treatment and/or prevention of conditions of total or relative iron deficiency, particularly for use in the treatment of disorders or diseases related to or resulting from iron deficiency.

It is an object of the present invention a composition (CC-1 or CC-2) comprising a mixture (CM-1 or CM-2) as described above and, optionally, at least one excipient and/or vehicle of pharmaceutical or food grade; preferably said composition is in solid form in oral dosage units.

It is also an object of the invention a composition CC-1 that comprises, or in the alternative consists of, the mixture CM-1, together with at least one excipient and/or vehicle of pharmaceutical or food grade.

It is also an object of the invention a composition CC-2 that comprises, or in the alternative consists of, the mixture CM-2, together with at least one excipient and/or vehicle of pharmaceutical or food grade.

The compositions CC-1 and CC-2 are suitable for oral administration.

Oral compositions CC-1 and CC-2 according to the invention are for use in conditions of total or relative iron deficiency, particularly for use in the treatment of disorders or diseases related to or resulting from iron deficiency.

The oral compositions CC-1 and CC-2 of the present invention preferably do not contain a diglycerol fatty acid.

The oral compositions CC-1 and CC-2 according to the invention are preferably a solid-state composition formulated in dosage units. Solid state means that the composition can exist in the form of granules or powders. The granular or powder compositions are then mixed with pharmacologically acceptable additives and excipients to provide an end product such as a supplement product, medical device, or pharmaceutical composition. The final product may be in pharmaceutical dosage units such as granules sachet, tablet, or capsule.

Tablets can have different shapes among those known in the field of pharmaceutical forms, such as a cylindrical or spheroidal shape. Tablets can range in weight from 100 to 2000 mg. Tablets may be coated or filmed with one or more layers of coating or film capable of passing through the gastric barrier, according to known methods.

For example, gel capsules may weigh 200-1200 mg, a hard capsule may weigh between 500 and 1000 mg, and a chewable tablet may weigh between 500 and 2000 mg. Capsules may be made of hard gelatin or soft gelatin or soft gel.

Preferably the oral composition of the invention is a solid composition, as described above. However, if desired or necessary, the composition may be formulated in liquid form, for example, as a suspension in water, preferably added with a physiologically acceptable acid, for example, citric acid.

The solid compositions of the invention may contain, as mentioned, physiologically acceptable conventional excipients and vehicles, such as diluents, bulking agents, binders, disaggregants, flow aids, lubricants, etc. Non-limiting examples of suitable vehicles and excipients are described in “Remington: The Science and Practice of Pharmacy, 21st Ed., Lippincott, Williams & Wilkins”. For example, the compositions of the invention may include cellulose derivatives, glucose, lactose, sucrose, gelatin, malt, rice, flour, gypsum, silica gel, sodium stearate, glycerol monostearate, talc, sodium chloride, skim milk powder, glycerol, propylene, glycol, water, ethanol, and the like. The composition may also contain pH buffering reagents and wetting or emulsifying agents.

In addition to the components described above, if desired or necessary, the compositions of the invention may include additional active components, for example, components that can assist the active ingredient in carrying out its action in the organism of the treated subject, such as vitamins.

The composition for oral use, solid or liquid, according to any of the embodiments described above, is useful in the treatment and prevention of disorders or diseases related to iron deficiency in pediatric subjects, adolescents, athletes, men, women, pregnant women, and the elderly because it prevents and counteracts anemia and is useful in increasing hemoglobin and ferritin values. Said composition is suitable for administration over a period from 1 to 5 months, preferably from 2 to 4 months, in pediatric subjects, adolescents, athletes, men, women, pregnant women, and the elderly, at a dose from 10 to 40 mg iron(III)/day, preferably from 14 to 30 mg iron(III)/day, even more preferably 28 mg iron(III)/day.

It is also an object of the invention an iron supplement comprising a composition CC-1 or CC-2 of the invention, possibly together with other components, e.g., minerals and/or vitamins.

It is also an object of the invention a method for the treatment and/or prevention of disorders or diseases related to iron deficiency, which comprises administering a composition CC-1 or CC-2 to a subject in need according to the invention.

The compositions CC-1 and CC-2 of the invention comprise iron (III) pyrophosphate in an amount from 30 to 70%, preferably from 35 to 55%, even more preferably from 40 to 50% by weight, with respect to the total weight of the composition.

The Applicant surprisingly found that the addition of a sodium or potassium pyrophosphate salt, e.g., a tetrasodium pyrophosphate, to the compositions described in WO2014/009806 and WO2015/033216 and marketed under the trade name “Sideral®”, completely unexpectedly, significantly increases iron up-take through increased ferritin.

This surprising result will be described and demonstrated in detail in the Experimental Section that follows. Sodium pyrophosphate is present preferably as tetrasodium pyrophosphate and is present, in the oral composition CC-1 or CC-2 of the present invention, in an amount from 0.1 to 30%, preferably from 1 to 20%, even more preferably from 12 to 16% by weight, with respect to the total weight of the composition.

The oral composition CC-1 of the present invention therefore comprises or alternatively consists of iron (III) pyrophosphate, a lecithin, for example a lecithin of the type E322, at least one gum arabic (or acacia gum) and sodium pyrophosphate, preferably tetrasodium pyrophosphate, in the above percent amounts by weight.

The oral composition CC-2 of the present invention therefore comprises or alternatively consists of iron (III)-pyrophosphate, a lecithin, for example a lecithin of the type E322, at least one gum arabic (or acacia gum), sodium pyrophosphate, preferably tetrasodium pyrophosphate, and iron (III)-sodium pyrophosphate, in the above percent amounts by weight.

As mentioned, the oral composition CC-1 or CC-2 of the present invention may also include other physiologically acceptable conventional components, excipients, and vehicles.

Preferably, the oral composition CC-1 or CC-2 of the present invention may additionally include an additional component selected from plant starches.

Plant starch is preferably selected from rice starches or corn starches. Preferably, the starch is rice starch; more preferably, rice starch is gelatinized or pregelatinized native rice starch.

A pregelatinized rice starch that can be used within the scope of the present invention may have the following physicochemical characteristics: moisture content not more than 7%; protein content not more than 1%; ash content not more than 1%; pH (10% solution) between 5.5 and 7.5; density 0.40-0.48 g/cm³; starch content not less than 97% and fat content not more than 0.1%. For example, pregelatinized rice starch can be used.

Gelatinized or pregelatinized plant starch is present in the solid composition in an amount from 10 to 40%, preferably from 15 to 35%, even more preferably from 20 to 30% by weight, with respect to the total weight of the compositions CC-1 and CC-2.

The oral composition CC-1 of the present invention comprises or, alternatively, consists of iron (III) salts, a lecithin, for example a lecithin type E322, at least one gum arabic (or one acacia gum), sodium pyrophosphate preferably tetrasodium pyrophosphate, and, preferably, a plant starch, in the above percent amounts by weight.

The oral composition CC-2 of the present invention comprises or alternatively consists of salts of iron (III), a lecithin, for example, a lecithin of the type E322, at least one gum arabic (or one acacia gum), sodium pyrophosphate preferably tetrasodium pyrophosphate, iron (III)-sodium pyrophosphate, and preferably a plant starch, in the above percent amounts by weight.

It is also an objectaid of the present invention a first method for preparing an oral composition CC-1 or CC-2 of the present invention.

A first method of the present invention is directed to the preparation of an oral composition comprising or, alternatively, consisting of iron (III)-pyrophosphate, a sunflower lecithin, gum arabic, tetrasodium pyrophosphate, a plant starch, preferably pregelatinized rice starch, and possibly iron (III)-sodium pyrophosphate, in the above percent amounts by weight.

Said first method of the present invention comprises or, alternatively, consists of a series of processing steps by which iron (III) pyrophosphate is coated or wrapped or encapsulated with said lecithin and/or said gum arabic and/or said plant starch.

Preferably, iron (III) pyrophosphate in the solid state is brought into contact, in order, with said gum arabic, then with said lecithin, then with said tetrasodium pyrophosphate, and finally with said plant starch.

Iron (III) pyrophosphate, lecithin, gum arabic, tetrasodium pyrophosphate, possibly iron (III)-sodium pyrophosphate and starch used in the method of the invention have the characteristics and properties defined above.

Starch in the form of gelatinized or pregelatinized starch has the advantage of being more fluid and free-flowing, allowing it to be accurately dosed without causing errors or weight variations. It also distributes more evenly and homogeneously. Finally, pregelatinized starch improves the bioavailability of the iron cation, as the resulting compound dissolves better at temperatures ranging from 15 to 30° C. (pressure 1 atmosphere), preferably from 20 to 25° C., even more preferably from 18 to 23° C.

Upon completion of said first method of preparation, an oral composition CC-1 or CC-2 of the present invention is obtained, which comprises or alternatively consists of iron (III) pyrophosphate, a sunflower lecithin, gum arabic, tetrasodium pyrophosphate, possibly iron (III)-sodium pyrophosphate (CC-2), and a pregelatinized rice starch, in the above percent amounts by weight.

Specifically, by said first method of preparation, a solid composition CC-1 or CC-2 of the present invention is obtained, which comprises or alternatively consists of iron (III) pyrophosphate, a sunflower lecithin E322, gum arabic, tetrasodium pyrophosphate, possibly iron (III)-sodium pyrophosphate (CC-2), and a pregelatinized rice starch, in the above percent amounts by weight.

The Applicant has found that to further improve the absorption of iron cation, it is useful to reduce the amount by weight of lecithin to be used in the process to prepare the solid composition of the present invention as much as possible.

In addition, the Applicant has found that to further enhance iron cation absorption, it is important to use a specific amount by weight of gum arabic in combination with a reduced amount by weight of lecithin.

Advantageously, the weight ratio of gum arabic to lecithin is between 40:1 and 10:1. In an embodiment said ratio is between 35:1 and 15:1; in each case lecithin is present in the composition in an amount by weight from 0.01% to 10%, preferably from 0.1% to 5%, more preferably from 0.5% to 2.5%, even more preferably from 0.8% to 1.2%.

Details of the first method of the invention are given in the Experimental Section of the present invention.

It is also an objectaid of the present invention a second method for preparing an oral composition CC-1 or CC-2 of the present invention.

A second method of the present invention is directed to the preparation of a solid composition CC-1 or CC-2 comprising or, alternatively, consisting of an iron salt, gum arabic, a lecithin, tetrasodium pyrophosphate, possibly iron (III)-sodium pyrophosphate (CC-2) and a gelatinized or pregelatinized starch.

Said second method of the present invention comprises or, alternatively, consists of a technology developed to create a coating or encapsulation around iron so as to improve the stability and bioavailability of the cation.

In practice, said second method involves the formation of agglomerates or granules comprising iron (III) pyrophosphate, gum arabic, lecithin, tetrasodium pyrophosphate, possibly iron (III)-sodium pyrophosphate, and a gelatinized or pregelatinized starch. All these components have the above characteristics.

Gum arabic and lecithin act by promoting the absorption of salt and, consequently, of the iron cation contained in said salt. The mixture with lecithin and starch results in the formation of “chimeric” agglomerates capable of protecting and shielding from gastric acid the iron cation contained in the pyrophosphate salt.

Processing time is between 1 and 60 minutes, preferably between 10 and 50, even more preferably between 20 and 40 minutes.

The oral composition obtained by the methods of the present invention has a particle size (which is understood to be the average particle size measured by available equipment and techniques) of the type D50 of about 20.5 μm (μm micrometer, 10-6 meters); for example, it may have a particle size distribution of the type D10 of about 1.8 μm, D50 of about 20.5 μm, and D90 of about 108 μm.

The solid composition of the present invention has an iron (III) content between 60 mg/g and 140 mg/g, preferably between 80 mg/g and 120 mg/g, even more preferably between 90 and 110 mg/g.

Oral composition CC-1 or CC-2 according to the invention is for use in conditions of total or relative iron deficiency, particularly for use in the treatment of disorders or diseases related to or resulting from iron deficiency.

The oral composition CC-1 or CC-2 of the present invention comprises iron that is easily absorbed and effectively bioavailable, and able to increase ferritin. In addition, oral composition CC-1 or CC-2 has been shown to be well tolerated by the body. The composition can be administered, even on an empty stomach, to all categories of patients, including pregnant women. Oral composition CC-1 or CC-2 has good palatability and has been shown to be stable over time chemically and organoleptically i.e., no changes in color, odor, taste and/or flavor have been observed.

Compositions CC-1 and CC-2 according to the invention are a solid state composition formulated in dosage units. Solid state means that the composition can exist as granules or powders. The granular or powder compositions are then mixed with pharmacologically acceptable additives and excipients to provide an end product such as a supplement product, medical device, or pharmaceutical composition.

The final product may be in pharmaceutical dosage units such as granules sachet, tablet, or capsule.

Tablets can have different shapes among those known in the field of pharmaceutical forms, such as a cylindrical or spheroidal shape. Tablets can range in weight between 200 and 2000 mg. Tablets may be coated or filmed with one or more layers of coating or film capable of passing through the gastric barrier, according to known methods.

For example, gel capsules may have a weight of 500 mg, a hard capsule may have a weight from 800 to 1000 mg, and a chewable tablet may have a weight from 1000 to 2000 mg. Capsules may be made of hard gelatin or soft gelatin or soft gel.

Each dosage unit includes from 5 to 50 mg of iron (III), preferably from 10 to 40 mg.

Preferably the oral composition CC-1 or CC-2 of the invention is a solid composition as described above.

However, if desired or necessary, the composition may be formulated in liquid form, for example as suspension in water, preferably added with a physiologically acceptable acid, e.g., citric acid.

The solid compositions of the invention may contain, as mentioned, physiologically acceptable conventional excipients and vehicles, such as diluents, bulking agents, binders, disaggregants, substances to promote flow, lubricants, etc. . . . Non-limiting examples of suitable vehicles and excipients are described in “Remington: The Science and Practice of Pharmacy, 21st Ed., Lippincott, Williams & Wilkins”.

For example, compositions of the invention may include cellulose derivatives, glucose, lactose, sucrose, gelatin, malt, rice, flour, gypsum, silica gel, sodium stearate, glycerol monostearate, talc, sodium chloride, skim milk powder, glycerol, propylene, glycol, water, ethanol, and the like. The composition may also contain pH buffering reagents and wetting or emulsifying agents.

Preferred excipients include hydroxypropyl methyl cellulose and magnesium salts of fatty acids.

In addition to the components described above, if desired or necessary, the compositions CC-1 and CC-2 of the invention may include additional active components, such as components that can assist the active ingredient in carrying out its action in the organism of the treated subject, such as vitamins.

It is also an objectaid of the present invention a liquid oral composition comprising or alternatively consisting of a composition CC-1 or CC-2 according to the invention, water and citric acid.

The composition CC-1 or CC-2 for oral use, solid or liquid, according to any of the above-described embodiments, is useful in the treatment and prevention of disorders or diseases related to iron deficiency in pediatric subjects, adolescents, athletes, men, women, pregnant women, and the elderly because it prevents and counteracts anemia and is useful in increasing hemoglobin and ferritin values. Said composition CC-1 or CC-2 is suitable for administration over a period from 1 to 5 months, preferably from 2 to 4 months, in pediatric subjects, adolescents, athletes, men, women, pregnant women, and the elderly, at a dose from 10 to 40 mg iron(III)/day, preferably from 14 to 30 mg iron(III)/day, even more preferably 28 mg iron(III)/day.

The compositions CC-1 and CC-2 can be administered throughout the pregnancy period, particularly from the 12^{th} weeks, up to 6 weeks postpartum. The recommended dose of iron (III) is from 10 to 40 mg/day, preferably from 14 to 30 mg/day, advantageously 28 mg/day.

It is also an objectaid of the present invention an iron supplement comprising a composition CC-1 or CC-2 of the invention, possibly together with other components, e.g., minerals and/or vitamins.

It is also an objectaid of the present invention a method for the treatment and/or prevention of disorders or diseases related to iron deficiency, which includes administering a composition CC-1 or CC-2 to a subject in need according to the invention.

The Applicant has conducted numerous experimental studies and has been able to observe that the mixtures and compositions of the invention, exhibit improved performance compared to commercially available compositions and even compared to compositions marketed under the trade name “Sideral*”.

Details of the assays conducted and results obtained are given in the Experimental Section below, which illustrates representative embodiments of the invention in non-limiting ways.

Embodiments of the present invention are given below.

Preferably, in the mixtures CM-1 and CM-2 and in compositions the CC-1 and CC-2 of the present invention, said i) an iron (III) pyrophosphate salt is present in said mixtures in an amount by weight from 10% to 90%, more preferably from 25% to 75%, more preferably from 35% to 65%, even more preferably from 40% to 55% by weight, with respect to the total weight of said mixtures.

Preferably, in the mixtures CM-1 and CM-2 and in the compositions CC-1 and CC-2 of the present invention, said salt i) iron (III) pyrophosphate salt is always present in an amount by weight, with respect to total weight, greater than 10% and less than 75%, by weight.

Preferably, the mixtures CM-1 and CM-2 and in compositions CC-1 and CC-2 of the present invention, are both in solid form, for example, as powders or granules, and are administered in the form of, for example, tablets, capsules, sachets, or sticks. The powders and granules, of mixtures CM-1 and CM-2 and in compositions CC-1 and CC-2 of the present invention, are produced by a process employing equipment and techniques known to the person skilled in the art, in that the individual components i)-ii)-iii) in the solid state are mixed and processed dry without the use of liquid solutions or water.

Preferably, in the mixtures CM-1 and CM-2 and in the compositions CC-1 and CC-2 of the present invention, the iron pyrophosphate is present in a weight ratio greater than 1, with respect to sodium pyrophosphate, preferably tetrasodium pyrophosphate ((FePP:NaPP)>1); or the iron pyrophosphate is present in a molar ratio greater than 1, with respect to sodium pyrophosphate, preferably tetrasodium pyrophosphate; or iron pyrophosphate is present in an equivalents ratio greater than 1 ((FePP:NaPP)>1 equivalent)).

Preferably, the weight ratio FePP:NaPP is from greater 1 to 15; more preferably said weight ratio is from 2 to 10; even more preferably said weight ratio is from 3 to 6, still even more preferably said weight ratio may be 3.5; or 4; or 4.5; or 5; or 5.5.

Preferably, the molar or millimolar ratio FePP:NaPP is from greater than 1 to 15; more preferably said molar or millimolar ratio is from 2 to 10; even more preferably said molar or millimolar ratio is from 3 to 6, still even more preferably said molar or millimolar ratio may be 3.5; or 4; or 4.5; or 5; or 5.5.

Preferably, the equivalents ratio FePP:NaPP is from greater 1 to 15; more preferably said equivalents ratio is from 2 to 10; even more preferably said equivalents ratio is from 3 to 6, still even more preferably said equivalents ratio may be 3.5; or 4; or 4.5; or 5; or 5.5.

It is an object of the present invention a process for the preparation of a mixture CM-1 and CM-2 and a composition CC-1 and CC-2, wherein said process comprises at least one step of mixing the individual components i) and/or ii) and/or iii) in the solid state to give said mixture and said composition in the solid state, preferably as powders or granules.

### BRIEF DESCRIPTION OF THE FIGURES

FIG. 1 shows a scheme of the static permeation assay.

FIG. 2 shows the results of comparison in solubility tests of Iron sulfate, Iron (III) pyrophosphate, Sideral r.m. (commercial product containing Iron (III) pyrophosphate, lecithin, sucroester and starch), Sideral NaPP (Sideral r.m. with added tetrasodium pyrophosphate according to the invention—Example 1).

FIG. 3 shows the results of comparison in the simulated gastric digestion test of Sideral r.m. (commercial product containing iron (III) pyrophosphate, lecithin, sucroester and starch), Sideral NaPP (Sideral r.m. with added tetrasodium pyrophosphate according to the invention—Example 1).

FIG. 4 shows the results of comparison in the ferritin accumulation test (Sideral_L ABS=composition of Example 1 of the invention with Lecithin ABS LecySpray; Sideral_NaPP_L AF NVH=composition of Example 1 of the invention with Lecithin L AF NVH; Sideral Liquid_NaPP_L AF NVH=composition of Example 2 with Lecithin L AF NVH).

FIG. 5 shows the results of the comparison in the apparent permeability and ferritin accumulation tests of Sideral with two different lecithins (L063 and LAFNVH) compared with the same Sideral added with tetrasodium pyrophosphate.

FIG. 6 shows the results of the comparison in the apparent permeability and ferritin accumulation tests of Sideral-like (composition containing gum arabic instead of sucroester CM1/CC1 and CM2/CC2) with two different lecithins (L063 and LAFNVH) compared with the same Sideral-like added with tetrasodium pyrophosphate.

FIG. 7 presents on the left A) the hematocrit values from T0 to T14; in the center B) the hemoglobin values from T0 to T14 and on the right C) the hemoglobin values from T0 to T14 with statistical value at T11 p=0.0073 and at T14 p<0.0001: Statistic: 2-way ANOVA, correction for multiple comparison, Sidak's test. T11 and T14: statistically significant 0.5 mg/Kg Sideral NaPP VS Vehicle NaPP. P value reported on graph.

FIG. 8 presents on the left A) hemoglobin values and on the right B) hematocrit values at T4-T0; T7-T0; T11-T0 and T14-T0: Statistics: 2-way ANOVA. Correction for multiple comparison, Sidak's test. T4-T0: statistically significant 0.5 mg/Kg Sideral NaPP VS Sideral RM; T7-T0; T11-T0: statistically significant 0.5 mg/Kg Sideral NaPP VS Vehicle NaPP; T14-T0: statistically significant 0.5 mg/Kg Sideral NaPP VS Vehicle NaPP. P value reported on the graph.

FIG. 9 shows other hematological parameters analyzed at the end of the experiment such as on the left A) MCH (mean corpuscular hemoglobin) and on the right B) MCV (mean corpuscular value).

FIG. 10 shows other hematological parameters analyzed at the end of the experiment such as on the left A) Ret-He (reticulocyte hemoglobin) and on the right B) RBC (red blood cells).

FIG. 11 shows on the left A) iron values in the bone marrow; in the center B) iron values in the liver; and on the right C) iron values in the spleen.

FIG. 12 shows on the left A) the liver mFTL values and on the right B) the spleen mFTL values.

FIG. 13 shows on the left A) hepcidin mRNA values; in the center B) Bmp6 mRNA values; and on the right C) Id1 mRNA values.

FIG. 14 shows on the left A) the values of Saa1 mRNA and on the right B) the values of Socs3 mRNA.

FIG. 15 shows on the left A) serum iron values and on the right B) TSat values.

Note that:
ID=Iron deficiency: iron in an amount less than 9 mg/Kg.
Vehicle Sideral RM=it is a matrix that contains sucroester, lecithin, and possibly starch, but does not contain iron.
Sideral NaPP=Sideral sodium pyrophosphate, is a mixture (or composition) of the present invention which contains Iron(III) pyrophosphate, sucroester, lecithin, and possibly starch, and which also contains, in addition, sodium pyrophosphate (preferably, in the form of tetrasodium pyrophosphate). Preferably, when starch is present in the mixture (or composition) of the present invention, together with Iron(III) pyrophosphate, sucroester and lecithin, sodium pyrophosphate replaces all or part of said starch in the preparation of said Sideral NaPP.
Vehicle NaPP=Sodium pyrophosphate, preferably tetrasodium pyrophosphate.
Sucrosomial® Iron or Sideral RM is a mixture (or composition) on the market that contains Iron(III) pyrophosphate, sucroester, lecithin, and possibly starch.

### EXPERIMENTAL SECTION

### Example 1

### Preparation of a Composition of the First Aspect of the Invention.

TABLE 1

| Ingredient | Amount in grams per 100 g | |
|---|---|---|
| Iron (III) Pyrophosphate DRPL Superfine | 44.76 | g |
| Sucroester | 17.14 | g |
| Sodium pyrophosphate | 14 | g |
| Lecithin AF NVH or Lecithin 063 | 0.58 | g |
| Rice starch | 23.52 | g |
| Total | 100 | g |

Mixing protocol

Preparation of 300 g of prototype following the relevant Master Formula with NOVINOX mixer per 100 g of finished product.
1) Add the following components:
   IRON (III) PYROPHOSPHATE
      +
   SUCROESTER
      +
   LECITHIN
      +
   SODIUM PYROPHOSPHATE
2) Mix for 30 minutes 8.7 rpm hourly rotation
3) Add: rice starch
4) Mix for 30 minutes, 8.7 rpm hourly rotation
5) Sieve automatically for about 10 minutes
6) Mix for 30 minutes, 8.7 rpm hourly rotation
7) Measure the yield at the end of the process

### Example 2

### Composition of the First Aspect of the Invention in Liquid Form

TABLE 2

| Ingredient | Amount g/2 ml | |
|---|---|---|
| Composition of Example 1 | 7.16 | g |
| Citric acid | 4.00 | g |
| Water | 1.984 | ml |

### Example 3

### Preparation of a Composition of the Third Aspect of the Invention, as in Example 1

TABLE 3

| Ingredient | Amount in grams per 100 g | |
|---|---|---|
| Iron (III) Pyrophosphate DRPL Superfine | 44.76 | g |
| Gum arabic | 17.14 | g |
| Sodium pyrophosphate | 14 | g |
| Lecithin AF NVH or Lecithin 063 | 0.58 | g |
| Rice starch | 23.52 | g |
| Total | 100 | g |

### Example 4

### Static Permeation Assay—Transwell System

### Experimental Protocol

### Material

Cells: Caco-2 (HTB-37™), ATCC.

Insert: TC insert, for 12-well plates, PET, transparent, pore size: 0.4 μm, Sarstedt.

Plate: 12 wells.

Complete medium: Dulbecco's Modified Eagle's Medium (DMEM—reduced glucose content, with 1000 mg/L glucose, and sodium bicarbonate, without L-glutamine and phenol red, liquid, sterile-filtered, suitable for cell culture) (87%), L-Glutamine solution 200 mM (2%), MEM Non-essential amino acid solution (100×) (1%), fetal bovine serum (10%).

Complete medium without FBS: Dulbecco's Modified Eagle's Medium ((DMEM—reduced glucose content, with 1000 mg/L glucose, and sodium bicarbonate, without L-glutamine and phenol red, liquid, sterile-filtered, suitable for cell culture) (87%), L-Glutamine solution 200 mM (2%), MEM Non-essential amino acid solution (100×) (1%).

DMEM: Dulbecco's Modified Eagle's Medium with phenol red pH indicator (DMEM-with 1000 mg/L glucose, and sodium bicarbonate, without L-glutamine and phenol red, liquid, sterile-filtered, suitable for cell culture).

CelLytic™ MT: Mammalian Tissue Lysis/Extraction Reagent, Sigma Aldrich.

MTT 1 mg/mL: 3,2,5-diphenyltetrazolium bromide, standard colorimetric assay for measuring the activity of enzymes that reduce MTT to formazan, giving the substance a blue/purple color. This staining indicates cell viability.

Alcohol Acid: 19 parts of 2 propanol: 1 part of HCl 2M.

TEER: Trans-Epithelial Electrical Resistance, measurement of the integrity and strength of the intestinal barrier at confluence of the cell monolayer.

Treatments: Iron (III) pyrophosphate, Sideral r.m., Composition of Example 1 (Sideral NaPP), Blank.

Supernatants of simulated gastro-intestinal digests.

### Cell Proliferation:

Place 12 inserts 0.4 μm in a 12-well plate. Replicate for 2 plates.
Plate in the Apical compartment 1 mL of Caco-2 cells in complete medium at a density of 0.5×10⁵ cells per insert. Cells should be plated at passages above 25.
Add 1.5 mL of complete medium to the basal compartment.
Keep the cells in an incubator at 37° C. with 5% CO₂.
Change the medium every other day for 21 days.
At day 21, the cells are ready for experimentation.

### Simulated Gastro-Intestinal Digestion:

Apply the simulated gastro-intestinal digestion protocol to the following samples: Blank (B), Iron (III) pyrophosphate (FeP), Sideral r.m. (SID), Sideral with sodium pyrophosphate (SID NaPP).

Oral compartment: 3.5 mL of a 2 mg/mL of aqueous solution of element iron (3.5 mL of water for blank) are kept at 37° C. for 5 min under stirring.

Gastric compartment: add 6.5 mL of SGF (Simulated gastric fluid: 0.2 g NaCl in 100 mL of water brought to pH 1.2 with concentrated HCl). Keep the solution at 37° C. under stirring for 2 h.

Intestinal Compartment: 10 mL of gastric digest is added to 1 mL of NaHCO3 1M and 4 mL of SIF (Simulated intestinal fluid: 0.68 g of anhydrous KH2PO4 in 100 mL H2O pH 7.5 with NaOH 1M). Keep the solution at 37° C. under stirring for 2 h.

Upon completion of the simulated gastro-intestinal digestion, centrifuge the samples 8000 rpm for 5 min and collect the supernatant. Quantify the supernatant with the ICP-OES instrument.

### Experimentation

Preparation treatments: dilute the supernatant of the gastro-intestinal digests with complete medium without FBS to a final concentration of 1 mg/mL.
1. Move the inserts to a clean 12 well plate and remove the medium from the apical compartment. 2 complete plates of 24 inserts are needed per experiment.
2. Wash the cells with 500 ml of PBS for 2 times.
3. Add 400 mL of treatment to the apical compartment and 1600 mL of complete medium without FBS to the basal compartment.
4. Measure the TEER for the 24 inserts at TO, that is, immediately after treatment.
5. Wait for 3 h of treatment in an incubator at 37° C.
6. At the completion of 3 h of treatment, measure the TEER for all 24 inserts and then take Apical and Basal.
7. Wash cells with 500 ml of PBS 2 times for both plates
8. To plate 1 (12 inserts) add 400 uL of CelLytic™ MT and shake for 10 min.
9. At the end of 10 min, take CelLytic™ MT from each insert and centrifuge at 8000 rpm for 5 min. Take out the supernatant and store the cell pellet.

From plate 1 obtain apical, basal, pellet, cell supernatant of each insert. From plate 2 obtain apical and basal.
10. For evaluation of barrier integrity, an additional parameter to TEER, in plate 2 add 400 uL of DMEM with phenol red indicator to the apical compartment of each insert. Add 1600 uL of complete medium without FBS to the basal compartment. Wait 1 h in an incubator at 37° C. The same procedure is applied to a triplicate insert that did not undergo treatment (Positive Control) and an empty triplicate insert without cells (Negative Control).
11. After 1 h take the basal and analyze the staining by spectrophotometer at the wavelength of 479 nm. The result is compared with the positive and negative control.
12. Remove the apical and wash the cells with 500 ml of PBS 2 times.
13. Add 400 mL of MTT to the apical compartment and 1600 mL of complete medium without FBS to the basal compartment. Wait 2h in an incubator at 37° C.
14. At the end of 2 h remove the liquid from the two compartments and add 400 mL in the Apical compartment and 1600 mL in the basal compartment of acid alcohol. Wait 24 h in an incubator at 37° C.
15. At the end of 24 h, read the obtained staining at the wavelength of 570 and 650 nm on the spectrophotometer and compare it to the positive control (viability cells on insert that did not receive treatment).

Through Plate 2, the cell monolayer integrity value and its viability are obtained. to be associated with TEER values at T0 and T3h.

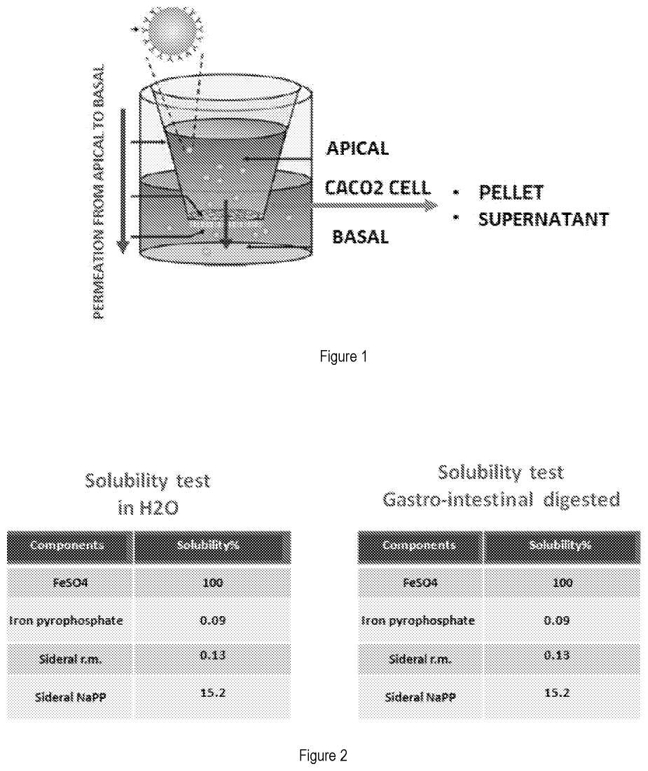

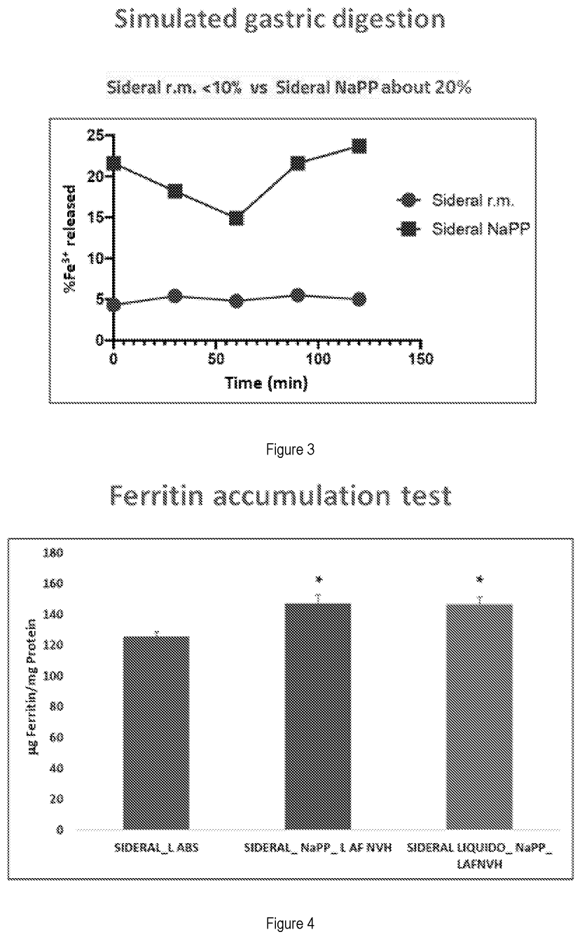

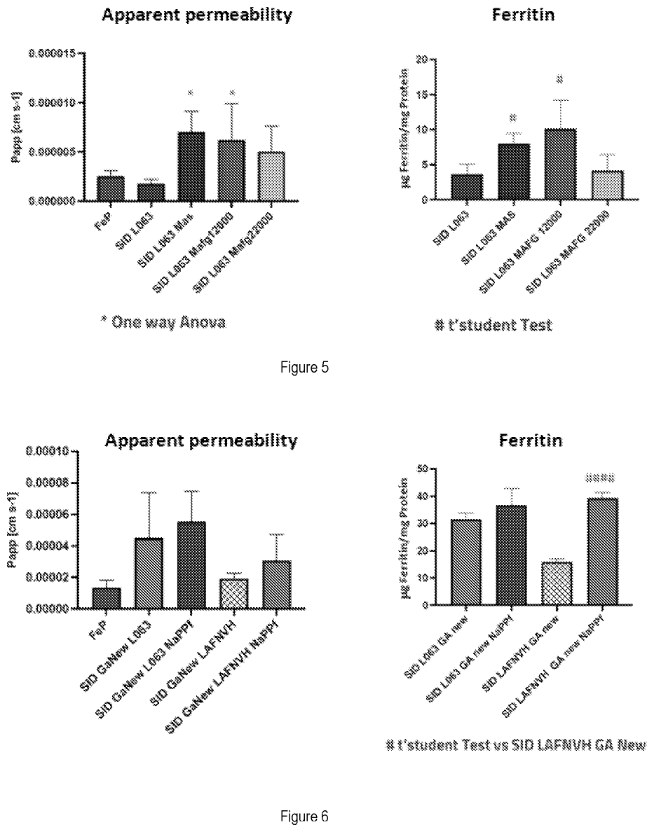

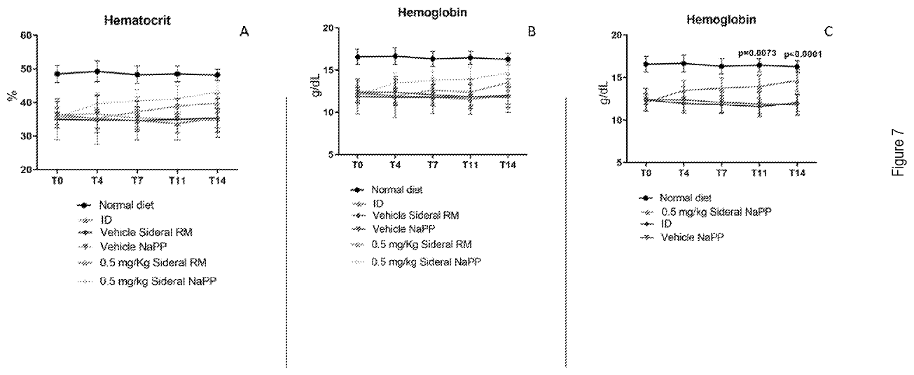

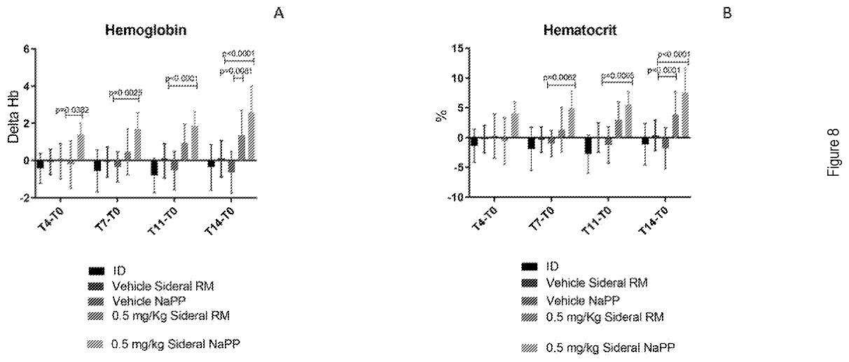

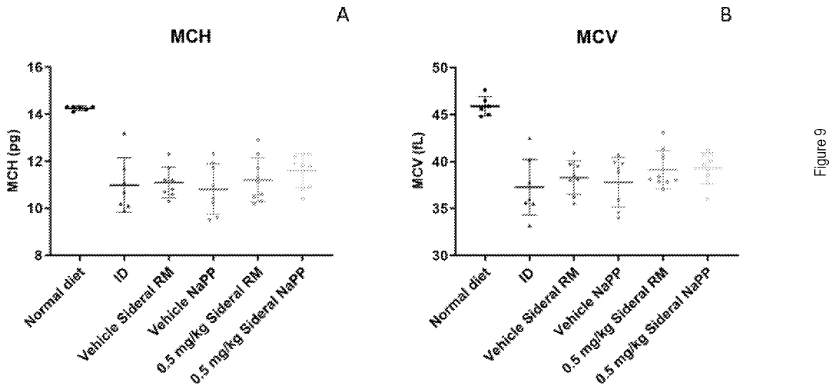

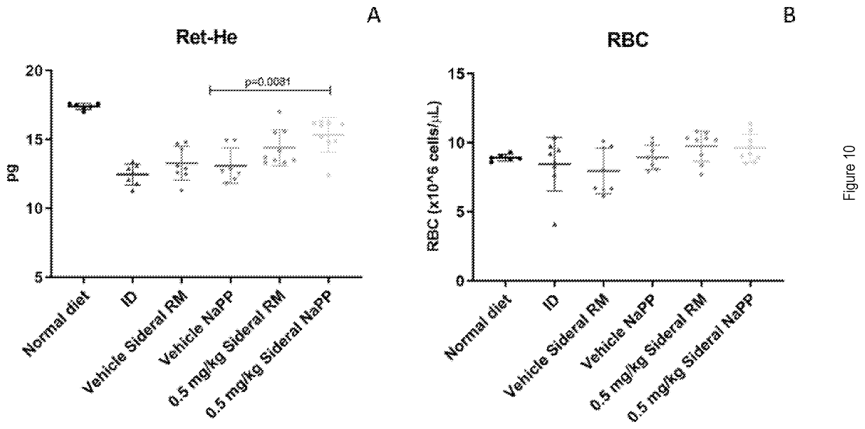

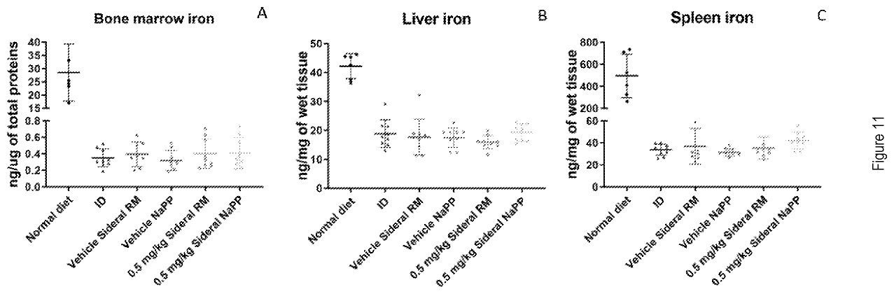

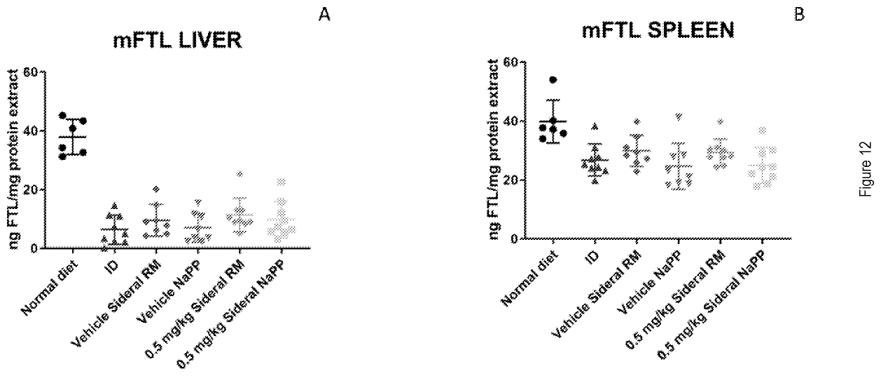

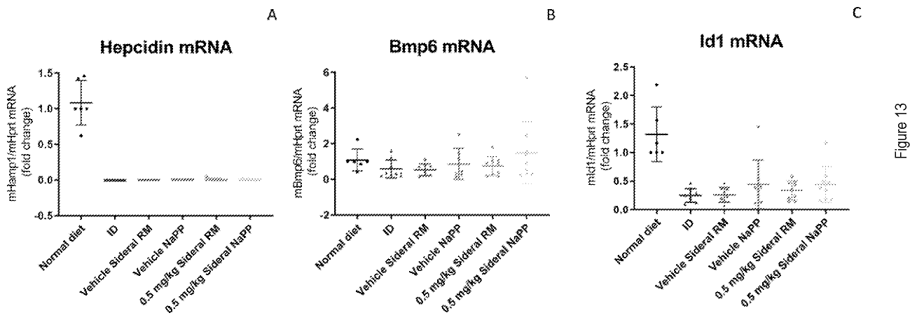

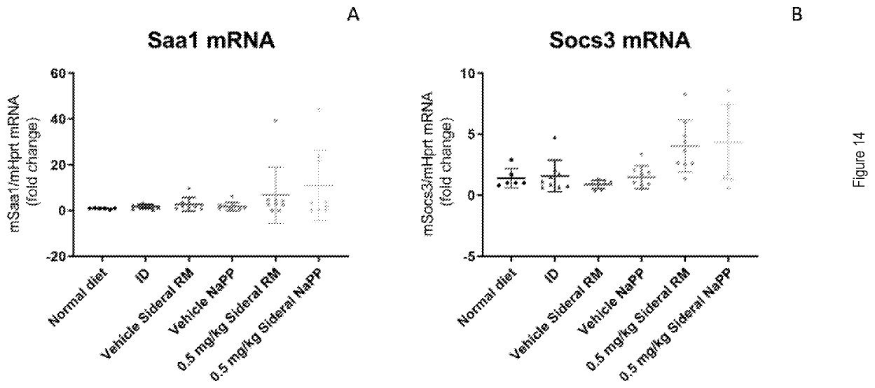

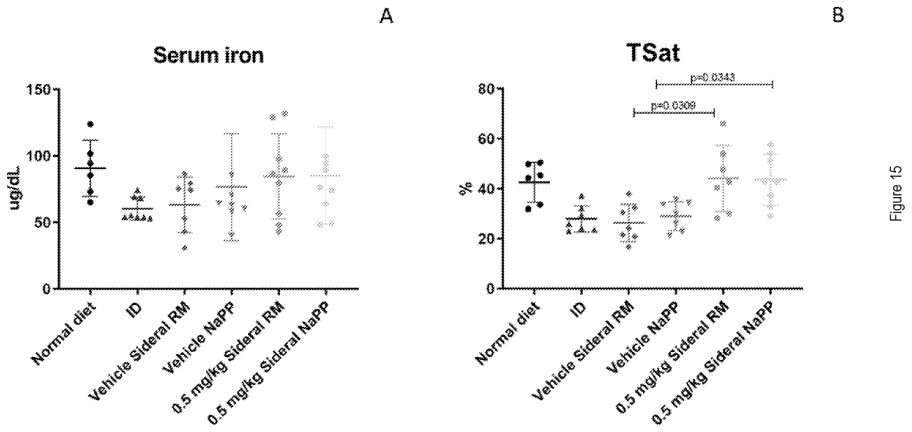

FIG. 1 shows a scheme of the static permeation system. The results of the assay are shown in FIGS. 2 and 3.

From the results shown in the Figures, it is understood that the composition according to any one of the aspects of the invention shows improved bioavailability of the iron contained therein and a surprising increase in ferritin. For these reasons, the composition according to any one of the aspects of the invention represents a significant technical advance over known iron-based compositions.

### Experimental Part

### Animals.

To induce iron-deficiency anemia, four-week-old male mice C57BL/J6 were placed on an iron-balanced diet or a low-iron diet containing <9 mg/kg of carbonyl iron (Code PF4418, from Mucedola s.r.l.) for at least 8 weeks. Hemoglobin and hematocrit were monitored weekly with the instrument Hemo_Vet (InfraTec, Dresden, Germany) by collecting a single drop of blood. Mice had basal Hb levels of 15.5-17 g/dL, and iron treatment was started when Hb fell below 12.5-13.0 g/dL. Treatment consisted of daily oral administration of vehicle (Vehicle Sideral RM, but without iron pyrophosphate), Sucrosomial® Iron (Sideral RM), vehicle NaPP (Vehicle NaPP) and Sideral NaPP, at a concentration of 0.5 mg/Kg of elemental iron, for two weeks. Mice were sacrificed two weeks after treatment. Blood and different tissues were collected for further analysis.

Note that:

ID=Iron deficiency: iron in an amount less than 9 mg/Kg.
Vehicle Sideral RM=it is a matrix that contains sucroester, lecithin, and possibly starch, but does not contain iron.
Sideral NaPP=Sideral sodium pyrophosphate, is a mixture (or composition) of the present invention that contains Iron(III) pyrophosphate, sucroester, lecithin, and possibly starch, and that also contains, in addition, sodium pyrophosphate (preferably, in the form of tetrasodium pyrophosphate). Preferably, when starch is present in the mixture (or composition) of the present invention, together with Iron(III) pyrophosphate, sucroester and lecithin, sodium pyrophosphate replaces all or part of said starch in the preparation of said Sideral NaPP.
Vehicle NaPP=Sodium pyrophosphate, preferably tetrasodium pyrophosphate.
Sucrosomial® Iron or Sideral RM is a mixture (or composition) on the market that contains Iron(III) pyrophosphate, sucroester, lecithin, and possibly starch.

### Analysis

### Hematological Analysis.

Hemoglobin and hematocrit were monitored with the instrument Hemo Vet (InfraTec, Dresden, Germany) at time 0 (T0) and at 4-7-11-14 days of treatment. At the end of the experiments, blood was collected and analyzed for red blood cells (RBCs), mean corpuscular value (MCV), mean corpuscular hemoglobin (MHC) and reticulocyte hemoglobin (Ret-He) (analysis performed by the IZLER Institute in Brescia, Italy).

### Serum Hepcidin and Iron.

Mouse serum hepcidin was quantified using a validated commercial Elisa kit (Cod. HMC-001) from Intrinsic Lifesciences respectively). Iron and transferrin saturation in serum was determined spectrophotometrically with commercial kits according to the manufacturer's instructions (Cod. MAK025 from Sigma-Aldrich and Cod. TI1010 from Randox Laboratories).

### Iron Quantification.

Tissue iron content was determined spectrophotometrically. Briefly, 50 mg of wet tissue was incubated for 18 h at 65° C. in 0.5 mL of HCl 3 M and trichloroacetic acid 0.6 M. After centrifugation, 10 μL of sample was added to 240 μL of working chromogen reagent, containing 1 vol. of 0.1% bathophenanthroline sulfonate/1% thioglycolic acid solution, 5 vol. of water and 5 vol. of saturated sodium acetate, in a 96-well plate. The samples were then incubated for 30 min at room temperature, and absorbance was measured at 535 nm in a plate reader. A standard curve was prepared with a pre-calibrated solution of FeCl3 (Sigma-Aldrich). The value obtained was normalized to the mg of wet tissue initially used for this analysis.

### ELISA for Mouse L-Ferritin.

L-ferritin was quantified by an in-house ELISA assay. The rabbit anti-mouse FtL antiserum was taken up on the 96-well microplate by adding 100 μL of 10 μg/mL in sodium carbonate 50 mM, pH 9.6 at 4° C. for 18 hours or 37° C. for 2 hours. After three washes with 200 μL of phosphate-buffered saline (PBS) with 0.1% Tween (PBST), the wells were covered by adding 100 μL of 3% bovine serum albumin (BSA) in PBS for 1 h at 37° C. After washing, 100 μL of 50 μg of liver and spleen protein extracts in PBST were added to the wells and incubated for 2 h at 37° C. After washing, 100 μL of HRP-conjugated anti-FtL antibody at 1:500 dilution was added and incubated for 1 h at 37° C. HRP activity was detected using 1 mg/mL of tetramethylbenzene (TMB) in dimethyl sulfoxide (DMSO) diluted 1:10 with phosphate-citrate buffer, pH 5 with the addition of fresh hydrogen peroxide at the final concentration of 0.006%, and the absorbance was read at 620 nm by MultiskanEx plate reader (Thermo). The reaction was stopped by adding sulfuric acid 1 N, and the absorbance was measured at 405 nm. The assay was calibrated using various dilutions of purified recombinant mouse L-ferritin.

### Ferritin Evaluation.

Liver and spleen homogenates were heated at 70° C. for 10 min to enrich ferritins. Samples (equivalent to 100 μg for liver and spleen of prewarmed protein) were loaded on PAGE nondenaturing 7.5% and run for 3 h at 160 V. The gels were washed with water and incubated in 2% ferrocyanide (Sigma-Aldrich) and 2% HCl for 1 h. To increase the signal, the gels were incubated in 0.025% 3,30-diaminobenzidine (Sigma-Aldrich) and 0.05% H₂O₂ in TBE 1× for 15-60 min. The reaction was stopped by washing with tap water.

### Quantitative QRT-PCR.

Total RNA was isolated from tissues using TRIzol reagent (Ambion) according to the manufacturer's instructions. The cDNA was generated by reverse transcription using 1 μg of RNA and Improm-II reverse transcriptase (Promega) in 20 μL and analyzed by quantitative reverse transcription polymerase chain reaction (qRT-PCR) using SensiFAST SYBR Lo-ROX (Bioline) according to the manufacturer's instructions. All data were normalized to the expression of Hprt1 and expressed as relative quantification (2nd-ΔΔΔCt method).

The primers used are:

Hprt1:
For
5-CTGGTTAAGCAGTACAGCCAA-3,
Rev
5-CAGGAGGTCCTTTTCACCAGC-3;
Hepcidin:
For
5-AAGCAGGCAGACATTGCGAT-3,
Rev
5-CAGGATGTGGCTCTAGGCTATGT-3;
Socs3:
For
5-TTAAATGCCCTCTGTCCCAGG-3,
Rev
5-TGTTTGGCTCCTTGTGCC-3;
Saa1
For
5-AGAGGACATGAGGACACCAT-3;
Rev
5-CAGGAGGTCTGTAGTAATTGG-3;
Id1:
For
5-ACCCTGAACGGCGAGATCA-3,
Rev
5-TCGTCGGCTGGAACACATG-3.
Bmp6:
For
5-ATGGCAGGACTGGATCATTGC-3,
Rev
5-CCATCACAGTAGTTGGCAGCGT-3.

Statistics. For treatments, comparisons between vehicle and iron formulations were performed by two-way ANOVA with multiple comparisons adjusted by Sidak's test or ordinary one-way ANOVA with multiple comparisons adjusted by Tukey's test.

### In Vivo Experimental Plan (EXP3-2023)

### Experimental Groups: 2-Week Treatment

Male mice C57BL/6J were fed with an iron-deficient diet (<10 mg/Kg Fe). When the mice reached a hemoglobin level <12.5-13.0 g/dL
(T0), mice were randomly divided into 6 groups:
(a) mice in ID without any treatment (N=9 mice).
(b) Mice treated with Sideral RM vehicle (150 uL) daily by oral gavage (for 2 weeks) (N=8 mice)
(c) Mice treated with 0.5 mg/Kg of Sideral RM (150 uL) daily orally (for 2 weeks) (N=9 mice)
(d) Mice treated with NaPP vehicle (150 uL) daily orally (for 2 weeks) (N=8 mice).
(e) Mice treated with 0.5 mg/Kg of Sideral NaPP (150 uL) daily orally (for 2 weeks) (N=9 mice) Note that 6 male mice were maintained on a normal diet as a healthy control.

Analysis: hemoglobin, hematocrit, at time 0 (T0) and at T4-T7-T11-T14 days of treatment. Ret-He and other blood parameters (analysis performed by the IZLER Institute at the end of the experiment). Iron content in liver, spleen and bone marrow. Serum iron and transferrin saturation (TSat).

1. A mixture comprising or alternatively consisting of:
i) an iron (III) pyrophosphate salt;
at least one lecithin;
at least one gum arabic or acacia gum; and
iii) a sodium or potassium pyrophosphate salt.

2. The mixture according to claim 1, wherein said mixture further comprises ii) an iron (III)-sodium pyrophosphate salt; preferably said ii) an iron (III)-sodium pyrophosphate salt is present in said mixture in an amount by weight from 0.1% to 50%, preferably from 1% to 35%, more preferably from 5% to 30%, even more preferably from 10% to 25% by weight, with respect to the total weight of said mixture.

3. The mixture according to claim 1 or 2, wherein said mixture further comprises a starch, preferably said starch is a plant starch selected from the group comprising or alternatively consisting of rice starch, corn starch, sunflower starch, or soybean starch, more preferably said plant starch is a pregelatinized rice starch.

4. The mixture according to claim 3, wherein said starch is present in said mixture in an amount by weight from 1% to 50%, preferably from 10% to 40%, more preferably from 15% to 35%, even more preferably from 20% to 30% by weight, with respect to the total weight of said mixture.

5. The mixture according to any one of claims 1-4, wherein said i) an iron (III) pyrophosphate salt is present in said mixture in an amount by weight from 10% to 90%, preferably from 25% to 75%, more preferably from 35% to 65%, even more preferably from 40% to 55% by weight, with respect to the total weight of said mixture.

6. The mixture according to any one of claims 1-5, wherein said at least one lecithin is a plant lecithin, preferably said plant lecithin is selected from the group comprising or alternatively consisting of a sunflower, corn, soybean or rice lecithin; preferably said at least one lecithin is present in said mixture in an amount by weight from 0.1% to 10%, preferably from 0.5% to 5%, more preferably from 1% to 4%, even more preferably from 1.5% to 3.5% by weight, with respect to the total weight of said mixture.

7. The mixture according to any one of claims 1-6, wherein said gum arabic or acacia gum is present in said mixture in an amount by weight from 5% to 45%, preferably from 10% to 40%, more preferably from 15% to 35%, even more preferably from 20% to 30% by weight, with respect to the total weight of said mixture.

8. The mixture according to any one of claims 1-7, wherein said iii) sodium or potassium pyrophosphate salt, preferably in the form of tetrasodium pyrophosphate, is present in said mixture in an amount by weight from 0.1% to 50%, preferably from 1% to 35%, more preferably from 5% to 30%, even more preferably from 10% to 25% by weight, with respect to the total weight of said mixture.

9. The mixture according to any one of claims 1-8, wherein said i) an iron (III) pyrophosphate salt is present in said mixture in an amount by weight from 10% to 90%, more preferably from 25% to 75%, more preferably from 35% to 65%, even more preferably from 40% to 55% by weight, with respect to the total weight of said mixture.

10. The mixture according to any one of claims 1-9, wherein said salt i) iron (III) pyrophosphate salt is always present in an amount by weight, with respect to the total weight, greater than 10%.

11. The mixture according to any one of claims 1-10, wherein said mixture is in a solid form; preferably, said mixture is in the form of powders or granules.

12. The mixture according to any one of claims 1-11, wherein said mixture is administered in the form of tablets, capsules, sachets, or sticks.

13. The mixture according to any one of claims 1-12, wherein in said mixture the iron (III) pyrophosphate is present in a weight ratio FePP:NaPP ranging from greater than 1 to 5, preferably ranging from 2 to 3, with respect to sodium pyrophosphate, preferably tetrasodium pyrophosphate.

14. The mixture according to any one of claims 1-12, wherein in said mixture the iron(III) pyrophosphate is present in a molar ratio FePP:NaPP ranging from greater than 1 to 5, preferably ranging from 2 to 3, with respect to sodium pyrophosphate, preferably tetrasodium pyrophosphate.

15. The mixture according to any one of claims 1-12, wherein in said mixture the iron pyrophosphate is present in an equivalents ratio FePP:NaPP ranging from greater than 1 to 5, preferably ranging from 2 to 3, with respect to sodium pyrophosphate, preferably tetrasodium pyrophosphate.

16. The mixture according to any one of claims 1-15, wherein said mixture is for use in therapy as a medicament.

17. The mixture for use according to claim 16, wherein said mixture is for use in a method of treatment and/or prevention of conditions of total or relative iron deficiency, particularly for use in the treatment of disorders or diseases related to or resulting from iron deficiency.

18. A composition comprising a mixture comprising or alternatively consisting of a mixture according to any one of claims 1-15 and, optionally, at least one excipient and/or vehicle of pharmaceutical or food grade; preferably said composition is in solid form in oral dosage units.

19. The composition according to claim 18, wherein said composition is for use in therapy as a medicament.

20. The mixture for use according to claim 18 or 19, wherein said composition is for use in a method of treatment and/or prevention of conditions of total or relative iron deficiency, particularly for use in the treatment of disorders or diseases related to or resulting from iron deficiency.

21. A process for preparing a mixture according to at least one of claims 1 to 17, or a composition according to at least one of claims 18 to 20, wherein said process comprises at least one step of mixing individual components i) and/or ii) and/or iii) in a solid state to give said mixture and said composition in a solid state, preferably as powders or granules.


SEQUENCE LISTING (12 sequences)

SEQ ID NO: 1 - DNA, 23 bp, synthetic construct
  source 1..23: mol_type = other DNA
ctggttaagcagtacagccccaa

SEQ ID NO: 2 - DNA, 21 bp, synthetic construct
  source 1..21: mol_type = other DNA
caggaggtccttttcaccagc

SEQ ID NO: 3 - DNA, 21 bp, synthetic construct
  source 1..21: mol_type = other DNA
aagcagggcagacattgcgat

SEQ ID NO: 4 - DNA, 23 bp, synthetic construct
  source 1..23: mol_type = other DNA
caggatgtggctctaggctatgt

SEQ ID NO: 5 - DNA, 21 bp, synthetic construct
  source 1..21: mol_type = other DNA
ttaaatgccctctgtcccagg

SEQ ID NO: 6 - DNA, 20 bp, synthetic construct
  source 1..20: mol_type = other DNA
tgtttggctccttgtgtgcc

SEQ ID NO: 7 - DNA, 20 bp, synthetic construct
  source 1..20: mol_type = other DNA
agaggacatgaggacaccat

SEQ ID NO: 8 - DNA, 21 bp, synthetic construct
  source 1..21: mol_type = other DNA
caggaggtctgtagtaattgg

SEQ ID NO: 9 - DNA, 19 bp, synthetic construct
  source 1..19: mol_type = other DNA
accctgaacggcgagatca

SEQ ID NO: 10 - DNA, 19 bp, synthetic construct
  source 1..19: mol_type = other DNA
tcgtcggctggaacacatg

SEQ ID NO: 11 - DNA, 21 bp, synthetic construct
  source 1..21: mol_type = other DNA
atggcaggactggatcattgc

SEQ ID NO: 12 - DNA, 22 bp, synthetic construct
  source 1..22: mol_type = other DNA
ccatcacagtagttggcagcgt